SEQUENCE LISTING (15 sequences)

SEQ ID NO: 1 - DNA, 2188 bp, Aspergillus niger
atgcgtggctctcggttggtgctcttgttgcccctggctgcacttagttgtgctatgcccgagaatgaatggtcatctacgataagaaggcagttaccaaaagcgtccactggcgtcaaatcgataaaaaccccaaacaatgtcactatcaggtataaagaaccaggaaccgaaggaatttgtgagacaacacctggggtcaaatcatactccggatatgtcgatctttcgccagagtcgcatactttcttttggtttttcgagtcacgccgtgaccccgaaaatgatccagtgactctgtggctgaatggtggccctggaagcgattccttgattgggctttttgaaggttggccaaatatcctgacggaaaagataaaattcagcttgcatgttctgacgccttcacaacagagttgggtccgtgtcacatcacaccagagtacgaatcaatcatcaatcagtactcctggaacgaggtcaccaatcttcttttcttgtctcagcccctcggtgtgggtatggaatattgctgccttcatacatcctgagtacattgcttacggtcttatctgcgaagggttctcttacagtgaaaccgaggccgggtccttgaatccatttactggagccgtcgagaacgcctcctttgctggagttcagggtcgatacccagttattgatgccactatcatcggtaagttgtccggtttgactctcacctagcattctcctcaatgtcctactttacagacacgaccgatatcgctgcacgcgcaacctgggaggtgcttcagggcttcctcagtggcctgtcgcagctagattccgaagtcaagtccaaggagttcaacctgtggacagagagttacggagggtgagtgcaactttcataccagaccgacgtaagctgacttgatcaagacactatggaccagcggtaggttgtcttttctggttgcacacatattgatctaatgaccgaagttcttcaatcatttctacgagcaaaattcgaagatcgctagcggggaagtcaatggcgtccaactgaattttaactccctcgggattatcaacggcatcattgatgccgcgattcaggtatttagaaatgcagctcgcgcagaggctgcggcctagaaggacatcgctaaagtaattaataggcagactactacgcagactttgccgttaataatacatatggaatcaaagctgtaagtttaaatacacgtacatcgtggatttaagatcaaccgtgctcatgcttgctaggtcaatgacacagtgtacaactatatgaagttcgccaacacgatgccaaatggatgccaggatcaggtcgcttcgtgtaaattgaccaataggacctcgctttctgattatgctatatgtacagaagcagccaatatgtgcagggacaatgtcggtgagtggttctactgtttctctgcaggggtgcaatgatgaaggactttgctaagctgtcatgtacagaagggccttactaccagtttggcggccgtggcgtgtatgatattcggcacccctacaatgtaagtggcaaggataaggattgtactttccgaacagggacactgctcatatgtcaacgtaggacccgaccccgccgtcctactttgttgactacctcaagaaagactcagtcatggatgctatcggcgtggacattaactacaccgagtccagcggcgaagtatattatgcattccagcagaccggcgactttgtatggccgaatttcattgaggacctcgaagagatcctccaactccccgtacgcgtgtcgttgatctacggcgatgccgactatatctgtaactggttcggcggtcaggccatctcactcgcagttaactacccccatgcagctcagttccgtgcagcgggatacacacccatgacagtagatggggtcgaatacggtgagactcgcgagtatggcaacttttcgttcacccgcgtatatcaggctgggcacgaggttccatactatcaaccgatcgcagcgttgcagctgttcaaccgtactttatttggatgggatattgcagcgggtacaactcagatttggcccgaatatagcaccaacgggacatcgcaggctacacacacggagtcgttcgtgccactgtccacggcgtcgagtaccaccgtcaattag

SEQ ID NO: 2 - protein, 552 aa, Aspergillus niger
MRGSRLVLLLPLAALSCAMPENEWSSTIRRQLPKASTGVKSIKTPNNVTIRYKEPGTEGICETTPGVKSYSGYVDLSPESHTFFWFFESRRDPENDPVTLWLNGGPGSDSLIGLFEELGPCHITPEYESIINQYSWNEVTNLLFLSQPLGVGFSYSETEAGSLNPFTGAVENASFAGVQGRYPVIDATIIDTTDIAARATWEVLQGFLSGLSQLDSEVKSKEFNLWTESYGGHYGPAFFNHFYEQNSKIASGEVNGVQLNFNSLGIINGIIDAAIQADYYADFAVNNTYGIKAVNDTVYNYMKFANTMPNGCQDQVASCKLTNRTSLSDYAICTEAANMCRDNVEGPYYQFGGRGVYDIRHPYNDPTPPSYFVDYLKKDSVMDAIGVDINYTESSGEVYYAFQQTGDFVWPNFIEDLEEILQLPVRVSLIYGDADYICNWFGGQAISLAVNYPHAAQFRAAGYTPMTVDGVEYGETREYGNFSFTRVYQAGHEVPYYQPIAALQLFNRTLFGWDIAAGTTQIWPEYSTNGTSQATHTESFVPLSTASSTTVN

SEQ ID NO: 3 - DNA, 27 bp, Artificial
  synthetic primer
acagcactcgcgagacaatgtgttatc

SEQ ID NO: 4 - DNA, 24 bp, Artificial
  synthetic primer
cgggtgaacgaaaagttgccatac

SEQ ID NO: 5 - DNA, 26 bp, Artificial
  synthetic primer
tttccagtctagacacgtataacggc

SEQ ID NO: 6 - DNA, 24 bp, Artificial
  synthetic primer
acggagtcggaccaagacactaag

SEQ ID NO: 7 - DNA, 25 bp, Artificial
  synthetic primer
aggtggcgagtcgagggattcgtag

SEQ ID NO: 8 - DNA, 5157 bp, Artificial
  synthetic plasmid sequence
cgagacaatgtgttatcgctattattggcaaaatggccgcgagatctcttatgcagggttcggctccatcctcccccctcttcctatccagtcaatccgcctcggttattgaaggagatgctgagctgtttaactgacgcctcaccgatcaggccggaaatggtggcgggatacaacatcgtttccacacaatagtgcttgtctcctgcgatctgcatggcatgctaatctccgccagcatgtatcttctatccactggatatgaattttcctcccctcacaccatgtgggcctgggggttttccctcaaactttgtcgctcatgtaacgatgtatataaagccctgaggatggcatcccccacccatcggtcttttgctgaccgttctccttgaagaaattctcgagtggcttgtggtgcatgtatagatttaatcttcgagggttattaactaggtatagctgtgactaagtctgtccttgcattgaacaacacaccatgcgtggctctcggttggtgctcttgttgcccctggctgcacttagttgtgctatgcccgagaatgaatggtcatctacgataagaaggcagttaccaaaagcgtccactggcgtcaaatcgataaaaaccccaaacaatgtcactatcaggtataaagaaccaggaaccgaaggaatttgtgagacaacacctggggtcaaatcatactccggatatgtcgatctttcgccagagtcgcatactttcttttggtttttcgagtcacgccgtgaccccgaaaatgatccagtgactctgtggctgaatggtggccctggaagcgattccttgattgggctttttgaaggttggccaaatatcctgacggaaaagataaaattcagcttgcatgttctgacgccttcacaacagagttgggtccgtgtcacatcacaccagagtacgaatcaatcatcaatcagtactcctggaacgaggtcaccaatcttcttttcttgtctcagcccctcggtgtgggtatggaatattgctgccttcatacatcctgagtacattgcttacggtcttatctgcgaagggttctcttacagtgaaaccgaggccgggtccttgaatccatttactggagccgtcgagaacgcctcctttgctggagttcagggtcgatacccagttattgatgccactatcatcggtaagttgtccggtttgactctcacctagcattctcctcaatgtcctactttacagacacgaccgatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatatcgctgcacgcgcaacctgggaggtgcttcagggcttcctcagtggcctgtcgcagctagattccgaagtcaagtccaaggagttcaacctgtggacagagagttacggagggtgagtgcaactttcataccagaccgacgtaagctgacttgatcaagacactatggaccagcggtaggttgtcttttctggttgcacacatattgatctaatgaccgaagttcttcaatcatttctacgagcaaaattcgaagatcgctagcggggaagtcaatggcgtccaactgaattttaactccctcgggattatcaacggcatcattgatgccgcgattcaggtatttagaaatgcagctcgcgcagaggctgcggcctagaaggacatcgctaaagtaattaataggcagactactacgcagactttgccgttaataatacatatggaatcaaagctgtaagtttaaatacacgtacatcgtggatttaagatcaaccgtgctcatgcttgctaggtcaatgacacagtgtacaactatatgaagttcgccaacacgatgccaaatggatgccaggatcaggtcgcttcgtgtaaattgaccaataggacctcgctttctgattatgctatatgtacagaagcagccaatatgtgcagggacaatgtcggtgagtggttctactgtttctctgcaggggtgcaatgatgaaggactttgctaagctgtcatgtacagaagggccttactaccagtttggcggccgtggcgtgtatgatattcggcacccctacaatgtaagtggcaaggataaggattgtactttccgaacagggacactgctcatatgtcaacgtaggacccgaccccgccgtcctactttgttgactacctcaagaaagactcagtcatggatgctatcggcgtggacattaactacaccgagtccagcggcgaagtatattatgcattccagcagaccggcgactttgtatggccgaatttcattgaggacctcgaagagatcctccaactccccgtacgcgtgtcgttgatctacggcgatgccgactatatctgtaactggttcggcggtcaggccatctcactcgcagttaactacccccatgcagctcagttccgtgcagcgggatacacacccatgacagtagatggggtcgaatacggtgagactcg

SEQ ID NO: 9 - DNA, 3352 bp, Aspergillus niger
atggcatctaaggacagagatatcctcccggacgtgtaagttaccgatcgccaacagctgagtatcgtgcacccatctgttggtgtttctctaacaatcgctggcagggtcaagcctgttcattacaatgtgtctttgtttgaccttcagttcggtggctcatggggttataagggtaccgtcaaaatcgattccaaggtcaaccgtcccacaaaagagatcgtgttgaactcgaaggaaatcgaagtccaggatgccgaagtttttggaaatgacggttagtgacagagccacactccaatttctcctgatccatttgtaatccatgatcgaatgttgagtttgacaaattggttgcacttagggaccaagctggcaaaggcatctaatatcgcttacgacaccaaatcggaaagagtcacctttacctttgcagaagaaatactccccgcagatgttgtcctgtcaatcaatttcaccggtataatgaacaatgccatggctggattttcccgctccaagtacaagccggtggtggatccgaccgacgatactcccaaggacggagattcctattacatgcttagtacccaattcgaatcttgtgatgctcgtagggccttcccttgctttgatgaaccgaatttgaaggccacattcgacttcgagatcgaggttccccgaggtcaaacagcccttagcaacatgcctatcaagtctgagaggagcggcagcaggccagagctcaagttggtctcttttgaaactacacctgttatgagtacttacgtcagtatgcacgattacttgcttatgatcctcggcgctaactttggagatcctccgttcagcttctggcatgggcagtcggtgactttgaatatgttgaggcaatgactcaacgcaaataccaaggcaagagcatacctgttcgcgtgtatacgaccaagggcctcaaggagcaggctcgctttgctttggaatgtgcccatcgtacagtcgattacttctctgaaatctttgagatcgagtatccgttgcctaaggcggatctccttgctgtccatgaatttgtacggaaattgatcctgttgcacacgaagcgaatatgctaaccgcaaatactctaggctatgggtgccatggagaactgggggcttgttacctaccgaacgactgctgttcttttcgatgagggcaagtcagacaccagatacaagaatcgtattgcatatgtcgtcgcgcatggtttgtcgtcccttccaatctgagcggccttgttccttcttaacgggtgtaatctagaattggcgcaccaatggtttggaaacctcgtaacgatggattggtggaatgagttgtggctcaatgagggctttgcaacatgggttggctggcttgctgtggatcatttctatccaggtatccgatggccccaggcgcttacagaaccagaaaagctgacgattktcttgcaaatagaatggaacatctggtctcagtttgtcgtacgcaattctcgaggtaatgcagtcctgtgtggcagaatattgacttttgcataggctgagagtgtccaacaagcatttcaactcgattcactgcgagcttcccacccgatcgaggtgcccgtgaggaacgctctcgaagtcgatcaaatttttgatcatatcagctacctcaaaggaagctcggtcattcggatgctaagtgatcatcttggccgggaaacattcctgcgtggagtagccgcatatctcaaagctcatgcatatggcaagtacacatcgaagacgtatagggaatcgggcttgctaattactgacaacttcaggtaatgccaccactaatgatctttggagcgctctcagcaaagcgtctaatcaggatgttactagctttatggtgagttatctcgttgtacagactggactactattctgacttcattacgaaggatccctggattcgcaagattggcttccctgttgtcaccgtaaccgagcaagccggccaactcagtgttcgtcaaagccgcttcttgtcgacgggtgatgttaagcccgaggaagatgagacggcctggtggattccccttggtgtaaaatcaggacctaagatggccgacgtaaaacccggtgctcttgtttccaaggaagatacaatttgggggctcggacaagattcctattacaagctgaacaaagacctgtctggcttttaccgaaccaattaccctgctgatcggctggcgaaactcgctcagtctctggaactgctgagcaccgaggataagattggattgattggcgatgccgctgccctagctgtttctggcgacggatccactgctgctctattggctctcttagagggtttcaagggcgagaagaattatttgtgagtaactaattatgatgtccttcattcgttctcgggtttcaacaatccttatgcttccatggttggttcttaacgacttgcatagggtctggtcccaaatctcttccactattgcgaacttacgctctgtgttcgccctgaatgagtcggtagcagcaggactcaagaagtttgccctcgagctttcatcccctgctgctaacaaaatcggctgggaatttagctcggaggatgactatctcactatccagctccgtaagcttctaatcggaatggctggccgtgcgggccacaacgagtaagtctattaggagatgttgtacgcttttcttgcaaatctaacttttttgctagcataatatccgaggccgaacgacgatttgagctttggaaatcaggcagcgataaggatgctgtgcataccaacctccgctcagtgatctttagtatcgtgatttcagagggcggtcgtgaagaatacaatgctgtaaagcaagagtatctcaagaccgactccgttgacggcaaggagatttgcttaggagctcttggacgtactaaagatgctgagctggtaaaggattacttagattttgttttctcagacaaggttgccatccaggatattcacaatggcgcggcttcaatggctacgaatccctcgactcgccaccttctatgggattatatgaaagagaattgggcggccgttgaaactcgcttgtcagcgaacaatgtcgttttcgagcggtttgtgcgtatgggactgtccaaattcgcgaaccacgatatcgcagccgatattgcttcgtttttccgggagaaggacactggtgcgtacgatcgcgccctggtaatcgttgctgatagcatccgaacgaatgcacgctacaaggaaagagatgagaagcaggtcttagagtggcttcggggccatggctacgcttga

SEQ ID NO: 10 - protein, 881 aa, Aspergillus niger
MASKDRDILPDVVKPVHYNVSLFDLQFGGSWGYKGTVKIDSKVNRPTKEIVLNSKEIEVQDAEVFGNDGTKLAKASNIAYDTKSERVTFTFAEEILPADVVLSINFTGIMNNAMAGFSRSKYKPVVDPTDDTPKDGDSYYMLSTQFESCDARRAFPCFDEPNLKATFDFEIEVPRGQTALSNMPIKSERSGSRPELKLVSFETTPVMSTYLLAWAVGDFEYVEAMTQRKYQGKSIPVRVYTTKGLKEQARFALECAHRTVDYFSEIFEIEYPLPKADLLAVHEFAMGAMENWGLVTYRTTAVLFDEGKSDTRYKNRIAYVVAHELAHQWFGNLVTMDWWNELWLNEGFATWVGWLAVDHFYPEWNIWSQFVAESVQQAFQLDSLRASHPIEVPVRNALEVDQIFDHISYLKGSSVIRMLSDHLGRETFLRGVAAYLKAHAYGNATTNDLWSALSKASNQDVTSFMDPWIRKIGFPVVTVTEQAGQLSVRQSRFLSTGDVKPEEDETAWWIPLGVKSGPKMADVKPGALVSKEDTIWGLGQDSYYKLNKDLSGFYRTNYPADRLAKLAQSLELLSTEDKIGLIGDAAALAVSGDGSTAALLALLEGFKGEKNYLVWSQISSTIANLRSVFALNESVAAGLKKFALELSSPAANKIGWEFSSEDDYLTIQLRKLLIGMAGRAGHNDIISEAERRFELWKSGSDKDAVHTNLRSVIFSIVISEGGREEYNAVKQEYLKTDSVDGKEICLGALGRTKDAELVKDYLDFVFSDKVAIQDIHNGAASMATNPSTRHLLWDYMKENWAAVETRLSANNVVFERFVRMGLSKFANHDIAADIASFFREKDTGAYDRALVIVADSIRTNARYKERDEKQVLEWLRGHGYA

SEQ ID NO: 11 - DNA, 25 bp, Artificial
  synthetic primer
acccgacgtggtggtatgaatgctc

SEQ ID NO: 12 - DNA, 2495 bp, Aspergillus niger
acagcactcgcgagacaatgtgttatcgctattattggcaaaatggccgcgagatctcttatgcagggttcggctccatcctcccccctcttcctatccagtcaatccgcctcggttattgaaggagatgctgagctgtttaactgacgcctcaccgatcaggccggaaatggtggcgggatacaacatcgtttccacacaatagtgcttgtctcctgcgatctgcatggcatgctaatctccgccagcatgtatcttctatccactggatatgaattttcctcccctcacaccatgtgggcctgggggttttccctcaaactttgtcgctcatgtaacgatgtatataaagccctgaggatggcatcccccacccatcggtcttttgctgaccgttctccttgaagaaattctcgagtggcttgtggtgcatgtatagatttaatcttcgagggttattaactaggtatagctgtgactaagtctgtccttgcattgaacaacacaccatgcgtggctctcggttggtgctcttgttgcccctggctgcacttagttgtgctatgcccgagaatgaatggtcatctacgataagaaggcagttaccaaaagcgtccactggcgtcaaatcgataaaaaccccaaacaatgtcactatcaggtataaagaaccaggaaccgaaggaatttgtgagacaacacctggggtcaaatcatactccggatatgtcgatctttcgccagagtcgcatactttcttttggtttttcgagtcacgccgtgaccccgaaaatgatccagtgactctgtggctgaatggtggccctggaagcgattccttgattgggctttttgaaggttggccaaatatcctgacggaaaagataaaattcagcttgcatgttctgacgccttcacaacagagttgggtccgtgtcacatcacaccagagtacgaatcaatcatcaatcagtactcctggaacgaggtcaccaatcttcttttcttgtctcagcccctcggtgtgggtatggaatattgctgccttcatacatcctgagtacattgcttacggtcttatctgcgaagggttctcttacagtgaaaccgaggccgggtccttgaatccatttactggagccgtcgagaacgcctcctttgctggagttcagggtcgatacccagttattgatgccactatcatcggtaagttgtccggtttgactctcacctagcattctcctcaatgtcctactttacagacacgaccgatatcgctgcacgcgcaacctgggaggtgcttcagggcttcctcagtggcctgtcgcagctagattccgaagtcaagtccaaggagttcaacctgtggacagagagttacggagggtgagtgcaactttcataccagaccgacgtaagctgacttgatcaagacactatggaccagcggtaggttgtcttttctggttgcacacatattgatctaatgaccgaagttcttcaatcatttctacgagcaaaattcgaagatcgctagcggggaagtcaatggcgtccaactgaattttaactccctcgggattatcaacggcatcattgatgccgcgattcaggtatttagaaatgcagctcgcgcagaggctgcggcctagaaggacatcgctaaagtaattaataggcagactactacgcagactttgccgttaataatacatatggaatcaaagctgtaagtttaaatacacgtacatcgtggatttaagatcaaccgtgctcatgcttgctaggtcaatgacacagtgtacaactatatgaagttcgccaacacgatgccaaatggatgccaggatcaggtcgcttcgtgtaaattgaccaataggacctcgctttctgattatgctatatgtacagaagcagccaatatgtgcagggacaatgtcggtgagtggttctactgtttctctgcaggggtgcaatgatgaaggactttgctaagctgtcatgtacagaagggccttactaccagtttggcggccgtggcgtgtatgatattcggcacccctacaatgtaagtggcaaggataaggattgtactttccgaacagggacactgctcatatgtcaacgtaggacccgaccccgccgtcctactttgttgactacctcaagaaagactcagtcatggatgctatcggcgtggacattaactacaccgagtccagcggcgaagtatattatgcattccagcagaccggcgactttgtatggccgaatttcattgaggacctcgaagagatcctccaactccccgtacgcgtgtcgttgatctacggcgatgccgactatatctgtaactggttcggcggtcaggccatctcactcgcagttaactacccccatgcagctcagttccgtgcagcgggatacacacccatgacagtagatggggtcgaatacggtgagactcgcgagtatggcaacttttcgttcacccg

SEQ ID NO: 13 - DNA, 25 bp, Artificial
  synthetic primer
accgtaggtaggcagacttggctcc

SEQ ID NO: 14 - DNA, 3300 bp, Aspergillus niger
acccgacgtggtggtatgaatgctcatgcagtccgtggtcaatctccaatttatcgtccggtccgaccagcacttctttctgcccctccttcatctcttcgtccacgctcccgctccctttccggattgccctactcgacatacctacagtctacccgtagggtcaaacctgctgtattgtcacccgtttctgtaaggcactgttcatctgaaatccgcgcaatggcatctaaggacagagatatcctcccggacgtgtaagttaccgatcgccaacagctgagtatcgtgcacccatctgttggtgtttctctaacaatcgctggcagggtcaagcctgttcattacaatgtgtctttgtttgaccttcagttcggtggctcatggggttataagggtaccgtcaaaatcgattccaaggtcaaccgtcccacaaaagagatcgtgttgaactcgaaggaaatcgaagtccaggatgccgaagtttttggaaatgacggttagtgacagagccacactccaatttctcctgatccatttgtaatccatgatcgaatgttgagtttgacaaattggttgcacttagggaccaagctggcaaaggcatctaatatcgcttacgacaccaaatcggaaagagtcacctttacctttgcagaagaaatactccccgcagatgttgtcctgtcaatcaatttcaccggtataatgaacaatgccatggctggattttcccgctccaagtacaagccggtggtggatccgaccgacgatactcccaaggacggagattcctattacatgcttagtacccaattcgaatcttgtgatgctcgtagggccttcccttgctttgatgaaccgaatttgaaggccacattcgacttcgagatcgaggttccccgaggtcaaacagcccttagcaacatgcctatcaagtctgagaggagcggcagcaggccagagctcaagttggtctcttttgaaactacacctgttatgagtacttacgtcagtatgcacgattacttgcttatgatcctcggcgctaactttggagatcctccgttcagcttctggcatgggcagtcggtgactttgaatatgttgaggcaatgactcaacgcaaataccaaggcaagagcatacctgttcgcgtgtatacgaccaagggcctcaaggagcaggctcgctttgctttggaatgtgcccatcgtacagtcgattacttctctgaaatctttgagatcgagtatccgttgcctaaggcggatctccttgctgtccatgaatttgtacggaaattgatcctgttgcacacgaagcgaatatgctaaccgcaaatactctaggctatgggtgccatggagaactgggggcttgttacctaccgaacgactgctgttcttttcgatgagggcaagtcagacaccagatacaagaatcgtattgcatatgtcgtcgcgcatggtttgtcgtcccttccaatctgagcggccttgttccttcttaacgggtgtaatctagaattggcgcaccaatggtttggaaacctcgtaacgatggattggtggaatgagttgtggctcaatgagggctttgcaacatgggttggctggcttgctgtggatcatttctatccaggtatccgatggccccaggcgcttacagaaccagaaaagctgacgattktcttgcaaatagaatggaacatctggtctcagtttgtcgtacgcaattctcgaggtaatgcagtcctgtgtggcagaatattgacttttgcataggctgagagtgtccaacaagcatttcaactcgattcactgcgagcttcccacccgatcgaggtgcccgtgaggaacgctctcgaagtcgatcaaatttttgatcatatcagctacctcaaaggaagctcggtcattcggatgctaagtgatcatcttggccgggaaacattcctgcgtggagtagccgcatatctcaaagctcatgcatatggcaagtacacatcgaagacgtatagggaatcgggcttgctaattactgacaacttcaggtaatgccaccactaatgatctttggagcgctctcagcaaagcgtctaatcaggatgttactagctttatggtgagttatctcgttgtacagactggactactattctgacttcattacgaaggatccctggattcgcaagattggcttccctgttgtcaccgtaaccgagcaagccggccaactcagtgttcgtcaaagccgcttcttgtcgacgggtgatgttaagcccgaggaagatgagacggcctggtggattccccttggtgtaaaatcaggacctaagatggccgacgtaaaacccggtgctcttgtttccaaggaagatacaatttgggggctcggacaagattcctattacaagctgaacaaagacctgtctggcttttaccgaaccaattaccctgctgatcggctggcgaaactcgctcagtctctggaactgctgagcaccgaggataagattggattgattggcgatgccgctgccctagctgtttctggcgacggatccactgctgctctattggctctcttagagggtttcaagggcgagaagaattatttgtgagtaactaattatgatgtccttcattcgttctcgggtttcaacaatccttatgcttccatggttggttcttaacgacttgcatagggtctggtcccaaatctcttccactattgcgaacttacgctctgtgttcgccctgaatgagtcggtagcagcaggactcaagaagtttgccctcgagctttcatcccctgctgctaacaaaatcggctgggaatttagctcggaggatgactatctcactatccagctccgtaagcttctaatcggaatggctggccgtgcgggccacaacgagtaagtctattaggagatgttgtacgcttttcttgcaaatctaacttttttgctagcataatatccgaggccgaacgacgatttgagctttggaaatcaggcagcgataaggatgctgtgcataccaacctccgctcagtgatctttagtatcgtgatttcagagggcggtcgtgaagaatacaatgctgtaaagcaagagtatctcaagaccgactccgttgacggcaaggagatttgcttaggagctcttggacgtactaaagatgctgagctggtaaaggattacttagattttgttttctcagacaaggttgccatccaggatattcacaatggcgcggcttcaatggctacgaatccctcgactcgccacct

SEQ ID NO: 15 - DNA, 4771 bp, Artificial
  synthetic plasmid sequence
ctgagtatcgtgcacccatctgttggtgtttctctaacaatcgctggcagggtcaagcctgttcattacaatgtgtctttgtttgaccttcagttcggtggctcatggggttataagggtaccgtcaaaatcgattccaaggtcaaccgtcccacaaaagagatcgtgttgaactcgaaggaaatcgaagtccaggatgccgaagtttttggaaatgacggttagtgacagagccacactccaatttctcctgatccatttgtaatccatgatcgaatgttgagtttgacaaattggttgcacttagggaccaagctggcaaaggcatctaatatcgcttacgacaccaaatcggaaagagtcacctttacctttgcagaagaaatactccccgcagatgttgtcctgtcaatcaatttcaccggtataatgaacaatgccatggctggattttcccgctccaagtacaagccggtggtggatccgaccgacgatactcccaaggacggagattcctattacatgcttagtacccaattcgaatcttgtgatgctcgtagggccttcccttgctttgatgaaccgaatttgaaggccacattcgacttcgagatcgaggttccccgaggtcaaacagcccttagcaacatgcctatcaagtctgagaggagcggcagcaggccagagctcaagttggtctcttttgaaactacacctgttatgagtacttacgtcagtatgcacgattacttgcttatgatcctcggcgctaactttggagatcctccgttcagcttctggcatgggcagtcggtgactttgaatatgttgaggcaatgactcaacgcaaataccaaggcaagagcatacctgttcgcgtgtatacgaccaagggcctcaaggagcaggctcgctttgctttggaatgtgcccatcgtacagtcgattacttctctgaaatctttgagatcgagtatccgttgcctaaggcggatctccttgctgtccatgaatttgtacggaaattgatcctgttgcacacgaagcgaatatgctaaccgcaaatactctaggctatgggtgccatggagaactgggggcttgttacctaccgaacgactgctgttcttttcgatgagggcaagtcagacaccagatacaagaatcgtattgcatatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatatatggcaagtacacatcgaagacgtatagggaatcgggcttgctaattactgacaacttcaggtaatgccaccactaatgatctttggagcgctctcagcaaagcgtctaatcaggatgttactagctttatggtgagttatctcgttgtacagactggactactattctgacttcattacgaaggatccctggattcgcaagattggcttccctgttgtcaccgtaaccgagcaagccggccaactcagtgttcgtcaaagccgcttcttgtcgacgggtgatgttaagcccgaggaagatgagacggcctggtggattccccttggtgtaaaatcaggacctaagatggccgacgtaaaacccggtgctcttgtttccaaggaagatacaatttgggggctcggacaagattcctattacaagctgaacaaagacctgtctggcttttaccgaaccaattaccctgctgatcggctggcgaaactcgctcagtctctggaactgctgagcaccgaggataagattggattgattggcgatgccgctgccctagctgtttctggcgacggatccactgctgctctattggctctcttagagggtttcaagggcgagaagaattatttgtgagtaactaattatgatgtccttcattcgttctcgggtttcaacaatccttatgcttccatggttggttcttaacgacttgcatagggtctggtcccaaatctcttccactattgcgaacttacgctctgtgttcgccctgaatgagtcggtagcagcaggactcaagaagtttgccctcgagctttcatcccctgctgctaacaaaatcggctgggaatttagctcggaggatgactatctcactatccagctccgtaagct

(19) United States
(12) Patent Application Publication
(10) Pub. No.: US 2009/0253173 A1
(43) Pub. Date: Oct. 8, 2009

(54) FILAMENTOUS FUNGI WITH INACTIVATED PROTEASE GENES FOR ALTERED PROTEIN PRODUCTION

(75) Inventor: Huaming Wang, Fremont, CA (US)

Correspondence Address: STEVEN G. BACSI;Danisco US Inc., Genencor Division, 925 PAGE MILL ROAD, PALO ALTO, CA, 94304-1013, US

(73) Assignee: Danisco US Inc., Genencor Division, Palo Alto, CA (US)

(21) Appl. No.: 12/412,297

(22) Filed: Mar. 26, 2009

Related U.S. Application Data
(60) Provisional application No. 61/043,284, filed on Apr. 8, 2008.

(51) Int. Cl. C12P 21/06; C12N 15/74; C12N 1/15; C12N 15/63; C07H 21/04

(52) U.S. Cl. 435/69.1; 435/471; 435/254.11; 435/254.3; 435/254.6; 435/254.8; 435/254.9; 435/320.1; 536/23.74

(57) ABSTRACT

The invention relates to a filamentous fungal cell (e.g., Aspergillus sp.) comprising at least one inactivated protease gene chosen from apsB, a homolog of apsB, cpsA, a homolog cpsA, and combinations thereof. Nucleic acids and methods for making the inactivated mutant filamentous fungal cells are provided as well as methods for using the cells for the altered production of endogenous or heterologous proteins of interest.

### CROSS-REFERENCE

The present application claims priority to U.S. Patent Application Ser. No. 61/043,284 filed on Apr. 8, 2008, which is hereby incorporated by reference in its entirety.

### FIELD OF THE INVENTION

The present invention relates to filamentous fungal microorganisms, such as Aspergillus species, engineered to have one or more inactivated protease genes so as to result in altered protein production.

### BACKGROUND OF THE INVENTION

Genetic engineering has allowed improvements in microorganisms used as industrial bioreactors, cell factories and in food fermentations. Important enzymes and proteins produced by engineered microorganisms include glucoamylases, α-amylases, cellulases, neutral proteases, and alkaline (or serine) proteases, hormones and antibodies. However, the occurrence of protein degradation and modification in some genetically engineered systems can interfere with efficient production.

Filamentous fungi (e.g., Aspergillus and Trichoderma species) and certain bacteria (e.g., Bacillus species) have been engineered to produce and secrete a large number of useful proteins and metabolites (see e.g., Bio/Technol. 5: 369-376, 713-719 and 1301-1304 [1987] and Zukowski, “Production of commercially valuable products,” In: Doi and McGlouglin (eds.) Biology of Bacilli: Applications to Industry, Butterworth-Heinemann, Stoneham. Mass pp 311-337 [1992]).

WO 97/22705, which is hereby incorporated by reference herein, relates to fungi, which do not produce certain proteases, and can be used as hosts for the production of proteins susceptible to proteolytic degradation by the proteases usually produced.

U.S. Pat. Nos. 5,840,570 and 6,509,171, each of which is hereby incorporated by reference herein, relate to mutant filamentous fungi which are deficient in a gene for an aspartic protein which are useful hosts for the production of heterologous polypeptides such as chymosin.

US patent application publication no. 2006/0246545, which is hereby incorporated by reference herein, relates to recombinant filamentous fungal cells having inactivated chromosomal genes corresponding to derA, derB, htmA, mnn9, mnn 10, ochA, dpp4, dpp5, pepAa, pepAb, pepAc, pepAd, pepF, and combinations thereof.

### SUMMARY OF THE INVENTION

In one embodiment, the invention provides a filamentous fungal cell comprising at least one inactivated gene, wherein the inactivated gene is chosen from apsB, a homolog of apsB, cpsA, a homolog cpsA, and combinations thereof. In some embodiments, the inactivated gene is a homolog of cpsA, wherein the homolog has at least 85% sequence identity to SEQ ID NO: 1, or encodes a polypeptide having at least 85% sequence identity to SEQ ID NO: 2. In some embodiments, the inactivated gene is a homolog of apsB, wherein the homolog has at least 85% sequence identity to SEQ ID NO: 9, or encodes a polypeptide having at least 85% sequence identity to SEQ ID NO: 10. In some embodiments, the inactivated gene is cpsA (SEQ ID NO: 1) or apsB (SEQ ID NO: 9).

In some embodiments, the inactivated gene encodes an intracellular protein, wherein the intracellular protein is involved in protein degradation and modification (e.g. protease genes, endoplasmic reticulum (ER) degradation pathway genes and glycosylation genes). In particular embodiments, the intracellular protein encoded by the inactivated gene is an N-terminal protease (e.g., an aminopeptidase such as apsB), or an intracellular C-terminal protease (e.g., carboxypeptidase).

In other embodiments, the filamentous fungal cell of the invention comprises an inactivated gene encoding a secreted protein, such as a protease.

In some embodiments, the filamentous fungal cells of the invention are cells from the filamentous fungi chosen from Aspergillus sp., Rhizopus sp., Trichoderma sp., and Mucor sp. In one aspect, the filamentous fungus is an Aspergilus sp. chosen from A. oryzae. A. niger, A. awamori, A. nidulans, A. sojae, A. japonicus, A. kawachi and A. aculeatus.

In some embodiments, the filamentous fungal cell comprises at least a first inactivated gene, chosen from apsB, a homolog of apsB, cpsA, a homolog cpsA, and a second inactivated gene chosen from apsB, cpsA, derA, derB, htmA, mnn9, mnn 10, ochA, dpp4, dpp5, pepAa, pepAb, pepAc, pepAd, pepB, pepC, pepD, pepF, and homologs thereof. In one aspect, the second inactivated gene is chosen from apsB, cpsA, dpp4, dpp5, and homologs thereof.

In some embodiments, the inactivated gene is inactivated by disruption with a selectable marker gene. Accordingly, in some embodiments the filamentous fungal cell further comprises a nucleic acid sequence encoding a selectable marker gene inserted in the nucleic acid sequence coding region of the inactivated gene. In some embodiments, the selectable marker gene is amdS.

In some embodiments, the filamentous fungal cell of the invention also produces an endogenous protein, wherein production of the endogenous protein by the cell is at least about 0% to about 200% (or more) greater than the production of the endogenous protein in a corresponding parent strain of the filamentous fungal cell. Accordingly, in some embodiments, the endogenous protein production is at least about 0% to 100% greater, in some embodiments is at least about 10% to 60% greater, including embodiments wherein production at least about 10%, 15%, 20%, 25%, 30%, 35%, 40%, 45%, 50%, and 55% greater, than the production of the endogenous protein in a corresponding parent strain of the filamentous fungal cell. In some embodiments, the endogenous protein is a glucogenic enzyme, or an enzyme chosen from α-amylase, cellulase, glucoamylase, laccase, neutral proteases, and alkaline protease.

In some embodiments, the filamentous fungal cell of the invention further comprises a nucleic acid encoding a heterologous protein. In some embodiments, the production of this heterologous protein is altered relative to the production of the same protein in a corresponding parent strain of the filamentous fungal cell. In some embodiments, the heterologous protein is an enzyme, and in particular an enzyme chosen from α-amylase, cellulase, glucoamylase, laccase, neutral proteases, and alkaline protease. In other embodiments, the heterologous protein is chosen from a protease inhibitor, antibody, or antibody fragment.

In some embodiments, the production of the heterologous protein by the filamentous fungal cell of the invention is at least about 0% to about 200% (or more) greater than the production of the heterologous protein in a corresponding parent strain of the filamentous fungal cell. Accordingly, in some embodiments, the heterologous protein production is at least about 0% to 100% greater, in some embodiments is at least about 10% to 60% greater, including embodiments wherein production at least about 10%, 15%, 20%, 25%, 30%, 35%, 40%, 45%, 50%, and 55% greater, than the production of the heterologous protein in a corresponding parent strain of the filamentous fungal cell. Additionally, in some embodiments, the total dry cell weight of the filamentous fungal cell of the invention differs by less than about 25%, 20%, 15%, 10%, 5%, or even less, than the total dry cell weight of a corresponding parent strain of the filamentous fungal cell.

In another embodiment, the invention provides a filamentous fungal cell comprising at least one inactivated gene, wherein the inactivated gene encodes an intracellular protein, and wherein production of at least one other protein by the cell is at least about 10% to 60% (including at least about 10%, 15%, 20%, 25%, 30%, 35%, 40%, 45%, 50%, and 55%) (or more) greater than the production of the other protein in a corresponding parent strain of the filamentous fungal cell. The other protein whose production is increased can be an endogenous (i.e., native) protein or a heterologous protein, and/or can be an intracellular or secreted protein.

In another embodiment, the present invention provides a filamentous fungal cell comprising at least one inactivated gene, wherein production of an endogenous and/or heterologous protein(s) of interest is at least about 0% to 100%, or even less than the production of the endogenous and/or heterologous protein in a corresponding parent strain of the filamentous fungus. In some embodiments, the production of the protein is at least about 10% to 60% less, including embodiments wherein production at least about 10%, 15%, 20%, 25%, 30%, 35%, 40%, 45%, 50%, and 55% less than the production of the endogenous and/or heterologous protein(s) in the corresponding parent strain.

In another embodiment, the present invention provides a method for producing a protein, wherein said method comprises: (a) introducing a nucleic acid encoding a protein into a filamentous fungal cell, wherein said cell comprises at least one inactivated gene, wherein the inactivated gene is chosen from apsB, a homolog of apsB, cpsA, a homolog cpsA, and combinations thereof; and (b) growing the cell under conditions suitable for producing the protein. In some embodiments, the method further comprises recovering the protein.

The methods for producing a protein of the invention can employ any fungal cell and, in some embodiments, any of the filamentous fungal cells as disclosed herein.

In another embodiment, the invention provides a method for making a filamentous fungal strain for protein production, wherein said method comprises: (a) transforming a filamentous fungal cell with a disruption sequence, wherein the disruption sequence comprises at least one inactivated gene chosen from apsB, a homolog of apsB, cpsA, a homolog cpsA, and combinations thereof; and (b) selecting the transformed cells wherein said disruption sequence is chromosomally integrated.

The methods for making a filamentous fungal strain for protein production of the invention can employ any of the filamentous fungal cell embodiments as disclosed herein.

In some embodiments of the method for making a filamentous fungal strain for protein production the disruption sequence comprises a selectable marker gene sequence reversely inserted at a restriction site in the coding region sequence of the inactivated gene. In some embodiments of this method, the selectable marker gene is amdS. In some embodiments, the restriction site comprises more than one site in the inactivated gene.

In some embodiments, the present invention provides linearized disruption plasmid fragment comprising a gene disruption sequence, wherein the gene is chosen from cpsA, a homolog of cpsA, apsB, and a homolog of apsB, and wherein the disruption sequence comprises a selectable marker gene sequence reversely inserted at a restriction site in the coding region sequence of the gene. In some embodiments, the gene is cpsA (SEQ ID NO: 1). In some embodiments, the gene is a homolog of cpsA, wherein the homolog has at least 85% sequence identity to SEQ ID NO: 1, or encodes a polypeptide having at least 85% sequence identity to SEQ ID NO: 2. In one embodiment, the invention provides a linearized disruption plasmid fragment wherein the disruption sequence has at least 95% identity (or more) to SEQ ID NO: 8. In some embodiments, the gene is apsB (SEQ ID NO: 9). In some embodiments, the gene is a homolog of apsB, wherein the homolog has at least 85% sequence identity to SEQ ID NO: 9, or encodes a polypeptide having at least 85% sequence identity to SEQ ID NO: 10. In one embodiment, the invention provides a linearized disruption plasmid fragment wherein the disruption sequence having at least 95% identity (or more) to SEQ ID NO: 15.

In another embodiment, the invention provides a vector comprising a disruption sequence of a gene, wherein the gene is chosen from cpsA, a homolog of cpsA, apsB, and a homolog of apsB, and wherein the disruption sequence comprises a selectable marker gene sequence reversely inserted at a restriction site in the coding region sequence of the gene. In one embodiment, the vector comprises a disruption sequence disruption sequence having at least 95% identity (or more) to SEQ ID NO: 8. In another embodiment, the vector comprises a disruption sequence disruption sequence having at least 95% identity (or more) to SEQ ID NO: 15.

In another aspect, the invention relates to a method of making a recombinant filamentous fungal cell comprising introducing into a filamentous fungal cell a DNA construct that recombines with a chromosomal gene chosen from apsB, cpsA, homologous sequences thereto and combinations thereof, whereby the chromosomal gene is inactivated. In some embodiments, the inactivated gene is disrupted and in other embodiments, the inactivated gene is deleted.

In another aspect, the invention relates to DNA constructs useful for generating inactivated mutants of filamentous fungal cell, wherein the DNA construct comprises a portion of the gene sequence of apsB, cpsA, homologous sequences thereto, and combinations thereof, disrupted by an intervening gene sequence.

### BRIEF DESCRIPTION OF THE DRAWINGS

FIG. 1 depicts the 2188 bp genomic DNA sequence of the Aspergillus niger cpsA gene (SEQ ID NO: 1).

FIG. 2 depicts the 552 amino acid sequence (SEQ ID NO: 2) encoded by the Aspergillus niger cpsA genomic DNA sequence of SEQ ID NO:1.

FIG. 3 depicts the DNA sequence of the W1 amplicon (SEQ ID NO:12) used in preparing inactivated cpsA strain.

FIG. 4 depicts plasmid map for the pBSΔcpsA-amd disruption plasmid that was linearized by Nrul restriction and used to transform Aspergillus niger resulting in the cpsA inactivated strain, ΔcpsA.

FIG. 5 depicts the DNA sequence (SEQ ID NO:8) of the portion of the pBSΔcpsA-amd disruption plasmid that was linearized by Nrul restriction and used to transform Aspergillus niger strain GICC2733.

FIG. 6 depicts image of electrophoretic gel showing the presence 1378 bp amplicon following PCR amplification of chromosomal DNA from the inactivated strain ΔcpsA. Lane 1: 100 bp DNA ladder marker; Lane 2: A. niger ΔcpsA strain chromosomal DNA used as amplification template; Lane 3: A. niger Δdpp4/Δdpp5 strain chromosomal DNA used as amplification template.

FIG. 7 depicts the 3352 bp genomic DNA sequence of the Aspergillus niger aminopeptidase gene, apsB (SEQ ID NO: 9).

FIG. 8 depicts the 881 amino acid sequence (SEQ ID NO: 10) encoded by the apsB genomic DNA sequence of SEQ ID NO:9.

FIG. 9 depicts the DNA sequence of the W2 amplicon (SEQ ID NO:14) used in preparing inactivated apsB strain.

FIG. 10 plasmid map for the pBSΔapsB-amdS disruption plasmid that was linearized by HindIII and PvuII restriction and used to transform Aspergillus niger resulting in the apsB inactivated strain, ΔapsB.

FIG. 11 depicts the DNA sequence (SEQ ID NO:15) for the portion of the pBSΔapsB-amdS disruption plasmid that was linearized by HindIII and PvuII restriction and used to transform Aspergillus niger strain GICC2733.

FIG. 12 depicts image of electrophoretic gel showing the presence 1604 bp amplicon following PCR amplification of chromosomal DNA from the inactivated strain ΔapsB. Lane 1: 100 bp DNA ladder marker; Lane 2: A. niger ΔapsB strain clone #28 chromosomal DNA used as amplification template; Lane 3: A. niger ΔapsB strain clone #87 chromosomal DNA used as amplification template; Lane 4: A. niger ΔapsB strain clone #93 chromosomal DNA used as amplification template; Lane 5: A. niger corresponding parent strain chromosomal DNA used as amplification template.

### DETAILED DESCRIPTION OF THE INVENTION

The present invention relates recombinant filamentous fungal cells, such as Aspergillus cells, having one or more inactivated protease genes. In some embodiments, the inactivated genes result an altered capacity of the filamentous fungal cells to produce other heterologous or endogenous proteins. In some embodiments, the cells having one or more inactivated genes produce a heterologous or endogenous protein in an amount at least about 10% to about 60% (or more) greater than the production of the same protein by the corresponding parent strain (i.e., non-inactivated strain) of the filamentous fungal cell.

### I. Definitions

All patents and publications, including all sequences disclosed within such patents and publications, referred to herein are expressly incorporated by reference. Unless defined otherwise herein, all technical and scientific terms used herein have the same meaning as commonly understood by one of ordinary skill in the art to which this invention belongs (See e.g., Singleton et al., DICTIONARY OF MICROBIOLOGY AND MOLECULAR BIOLOGY, 2 D ED., John Wiley and Sons, New York [1994]; and Hale and Marham, THE HARPER COLLINS DICTIONARY OF BIOLOGY, Harper Perennial, N.Y. [1991], both of which provide one of skill with a general dictionary of many of the terms used herein). Any methods and materials similar or equivalent to the various embodiments described herein can be used in the practice or testing of the present invention.

It is intended that every maximum (or minimum) numerical limitation disclosed in this specification includes every lower (or higher) numerical limitation, as if such lower (or higher) numerical limitations were expressly written herein. Moreover, every numerical range disclosed in this specification is intended to include every narrower numerical range that falls within such broader numerical range, as if such narrower numerical ranges were all expressly written herein.

As used herein, the singular “a”, “an” and “the” includes the plural reference unless the context clearly dictates otherwise. Thus, for example, reference to a “host cell” includes a plurality of such host cells.

Unless otherwise indicated, nucleic acids are written left to right in 5′ to 3′ orientation; amino acid sequences are written left to right in amino to carboxyl orientation, respectively. The headings provided herein are not limitations of the various aspects or embodiments of the invention that can be had by reference to the specification as a whole. Accordingly, the terms defined immediately below are more fully defined by reference to the Specification as a whole.

As used herein, the term “inactivation” refers to any method that substantially prevents the functional expression of one or more genes, fragments or homologues thereof, wherein the gene or gene product is unable to exert its known function. It is intended to encompass any means of gene inactivation include deletions, disruptions of the protein-coding sequence, insertions, additions, mutations, gene silencing (e.g. RNAi genes antisense) and the like. Accordingly, the term “inactivated” refers to the result of “inactivation” as described above. In some embodiments, “inactivation” will result in a cell having no detectable activity for the gene or gene product corresponding to the inactivated gene. In some embodiments, “inactivation” may result in little or no functional expression of a gene but still have functional expression of a homologue to the gene. Consequently, an “inactivated strain” may exhibit a partially active phenotype due to the homologue gene.

As used herein, an “inactivated mutant” or “inactivated strain” refers to a host organism (e.g., Aspergillus niger cells) having one or more inactivated genes. The term is intended to encompass progeny of an inactivated mutant or inactivated strain and is not limited to the cells subject to the original inactivation means (e.g., the initially transfected cells).

In some embodiments, “inactivation” is the result of gene deletions and these inactivated mutants are sometimes referred to as “deletion mutants.” In other embodiments, inactivation is the result of disruption to the protein coding sequence of a gene and these inactivated mutants are sometimes referred to as “disruption mutants.” In some embodiments, the inactivation is non-revertible.

As used herein, “deletion” of a gene refers to deletion of the entire coding sequence, deletion of part of the coding sequence, or deletion of the coding sequence including flanking regions.

As used herein “disruption” refers to a change in a nucleotide or amino acid sequence by the insertion of one or more nucleotides or amino acid residues, respectively, as compared to the parent or naturally occurring sequence. Accordingly, a “disruption sequence” or “disruption mutant” as used herein refers to a nucleic acid or amino acid sequence, typically a coding region sequence, that comprises an insertion of nucleotides or amino acids.

As used herein, “insertion” or “addition” in the context of a sequence refers to a change in a nucleic acid or amino acid sequence in which one or more nucleotides or amino acid residues have been added as compared to the endogenous chromosomal sequence or protein product.

As used herein, “non-revertable” refers to a strain which will naturally revert back to it corresponding parent strain with a frequency of less than 10⁻⁷.

As used herein, the term “corresponding parent strain” refers to the host strain from which an inactivated mutant is derived (e.g., the originating and/or wild-type strain).

As used herein, “strain viability” refers to reproductive viability. In some embodiments, the inactivation of a gene does not deleteriously affect division and survival of the inactivated mutant under laboratory conditions.

As used herein “coding region” refers to the region of a gene that encodes the amino acid sequence of a protein.

As used herein “amino acid” refers to peptide or protein sequences or portions thereof. The terms “protein,” “peptide,” and “polypeptide” are used interchangeably.

As used herein, the term “heterologous protein” or “exogenous protein” refers to a protein or polypeptide that does not naturally occur in the host cell, and includes genetically engineered versions of naturally occurring endogenous proteins.

As used herein, “endogenous protein” or “native protein” refers to a protein or polypeptide naturally occurring in a cell.

As used herein, “host,” “host cell,” or “host strain” refer to a cell that can express a DNA sequence introduced into the cell. In some embodiments of the present invention, the host cells are Aspergillus sp.

As used herein, “filamentous fungal cell” refers to a cell of any of the species of microscopic fungi that grow as multicellular filamentous strands including but not limited to: Aspergillus sp., Rhizopus sp., Trichoderma sp., and Mucor sp.

As used herein, “Aspergillus” or “Aspergillus sp.” includes all species within the genus “Aspergillus,” as known to those of skill in the art, including but not limited to A. niger, A. oryzae, A. awamori, A. kawachi and A. nidulans.

As used herein, “nucleic acid” refers to a nucleotide or polynucleotide sequence, and fragments or portions thereof, as well as to DNA, cDNA, and RNA of genomic or synthetic origin which may be double-stranded or single-stranded, whether representing the sense or antisense strand. It will be understood that as a result of the degeneracy of the genetic code, a multitude of nucleotide sequences may encode a given protein.

As used herein the term “gene” means a segment of DNA involved in producing a polypeptide and can include regions preceding and following the coding regions (e.g., promoter, terminator, 5′ untranslated (5′ UTR) or leader sequences and 3′ untranslated (3′ UTR) or trailer sequences, as well as intervening sequence (introns) between individual coding segments (exons).

As used herein, “homologous gene,” “gene homolog,” or “homolog” refers to a gene which has a homologous sequence and results in a protein having an identical or similar function. The term encompasses genes that are separated by speciation (i.e., the development of new species) (e.g., orthologous genes), as well as genes that have been separated by genetic duplication (e.g., paralogous genes).

As used herein, “homologous sequences” refers to a nucleic acid or polypeptide sequence having at least about 99%, at least about 98%, at least about 97%, at least about 96%, at least about 95%, at least about 94%, at least about 93%, at least about 92%, at least about 91%, at least about 90%, at least about 88%, at least about 85%, at least about 80%, at least about 75%, at least about 70% or at least about 60% sequence identity to a subject nucleotide or amino acid sequence when optimally aligned for comparison. In some embodiments, homologous sequences have between about 80% and 100% sequence identity, in some embodiments between about 90% and 100% sequence identity, and in some embodiments, between about 95% and 100% sequence identity.

Sequence homology can be determined using standard techniques known in the art (see e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

Useful algorithms for determining sequence homology include: PILEUP and BLAST (Altschul et al., J. Mol. Biol., 215:403-410, [1990]; and Karlin et al., Proc. Natl. Acad. Sci. USA 90:5873-5787 [1993]). PILEUP uses a simplification of the progressive alignment method of Feng and Doolittle (Feng and Doolittle, J. Mol. Evol., 35:351-360 [1987]). The method is similar to that described by Higgins and Sharp (Higgins and Sharp, CABIOS 5:151-153 [1989]). Useful PILEUP parameters including a default gap weight of 3.00, a default gap length weight of 0.10, and weighted end gaps.

A particularly useful BLAST program is the WU-BLAST-2 program (See, Altschul et al., Meth. Enzymol., 266:460-480 [1996]). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched. However, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

As used herein, the term “vector” refers to any nucleic acid that can be replicated in cells and can carry new genes or DNA segments into cells. Thus, the term refers to a nucleic acid construct designed for transfer between different host cells. An “expression vector” refers to a vector that has the ability to incorporate and express heterologous DNA fragments (i.e., non-native DNA) in a cell. Many prokaryotic and eukaryotic expression vectors are commercially available. Selection of appropriate expression vectors is within the knowledge of those having skill in the art.

As used herein, the terms “DNA construct,” “expression cassette,” and “expression vector,” refer to a nucleic acid molecule generated recombinantly or synthetically, with a series of specified nucleic acid elements that permit transcription of a particular nucleic acid in a target cell (i.e., vectors or vector elements, as described above). For example, an expression cassette can be incorporated into a plasmid, chromosome, mitochondrial DNA, plastid DNA, virus, or nucleic acid fragment. Typically, the expression cassette portion of an expression vector includes, among other sequences, a nucleic acid sequence to be transcribed, a promoter and a terminator. In some embodiments, DNA constructs also include a series of specified nucleic acid elements that permit transcription of a particular nucleic acid in a target cell. In some embodiments, a DNA construct of the invention comprises a selectable marker.

Also as used herein, the term “DNA construct” (as well as “transforming DNA,” and “transforming sequence”) refers to DNA that is used to introduce sequences into a host cell or organism (i.e., “transform a host cell”). The DNA construct may be generated in vitro by PCR or any other suitable techniques. In some embodiments, the transforming DNA can include an incoming sequence, and/or can include an incoming sequence flanked by homology boxes. In yet a further embodiment, the transforming DNA comprises other non-homologous sequences, added to the ends (e.g., stuffer sequences or flanks). The ends can be closed such that the transforming DNA forms a closed circle (i.e., a plasmid), such as, for example, insertion into a vector.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

As used herein, the terms “isolated” and “purified” are used to refer to a molecule (e.g., a nucleic acid or polypeptide) or other component that is removed from at least one other component with which it is naturally associated.

As used herein, the term “altered expression” is construed to include an increase or decrease in production of a protein of interest by an altered (i.e., engineered) cell strain relative to the normal level of production from the corresponding unaltered parent strain (i.e., when grown under essentially the same conditions).

As used herein, the term “enhanced expression” is construed to include the increased production of a protein of interest by an altered (i.e., engineered) cell strain above the normal level of production from the corresponding unaltered parent strain (i.e., when grown under essentially the same conditions).

As used herein, the term “expression” refers to a process by which a polypeptide is produced. The process includes both transcription and translation of the gene. In some embodiments, the process also includes secretion of the polypeptide.

As used herein in the context of “introducing a nucleic acid sequence into a cell,” the term “introducing” (and in past tense, “introduced”) refers to any method suitable for transferring the nucleic acid sequence into the cell, including but not limited to transformation, electroporation, nuclear microinjection, transduction, transfection, (e.g., lipofection mediated and DEAE-Dextrin mediated transfection), incubation with calcium phosphate DNA precipitate, high velocity bombardment with DNA-coated microprojectiles, agrobacterium mediated transformation, and protoplast fusion.

As used herein, the terms “stably transformed” refers to a cell that has a non-native (heterologous) polynucleotide sequence integrated into its genome or as an episomal plasmid that is maintained for at least two generations.

As used herein “an incoming sequence” refers to a DNA sequence that is being introduced into a host cell. The incoming sequence can be part of a DNA construct, can encode one or more proteins of interest (e.g., heterologous protein), can be a functional or non-functional gene and/or a mutated or modified gene, and/or can be a selectable marker gene(s). For example, the incoming sequence can include a functional or non-functional (e.g., disrupted) version of a gene chosen from apsB, cpsA, derA, derB, htmA, mnn9, mnn 10, ochA, dpp4, dpp5, pepAa, pepAb, pepAc, pepAd, pepF, pepB, pepC, pepD, fragments and homologous sequences thereof. In one embodiment, the incoming sequence includes two homology boxes.

As used herein, “homology box” refers to a nucleic acid sequence, which is homologous to the sequence of gene in the chromosome of a filamentous fungal cell. More specifically, a homology box is an upstream or downstream region having between about 80 and 100% sequence identity, between about 90 and 100% sequence identity, or between about 95 and 100% sequence identity with the immediate flanking coding region of a gene or part of a gene to be inactivated according to the invention. These sequences direct where in the chromosome a DNA construct or incoming sequence is integrated and directs what part of the chromosome is replaced by the DNA construct or incoming sequence. While not meant to limit the invention, a homology box may include between about 1 base pair (bp) to 200 kilobases (kb). Typically, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

In an alternative embodiment, the transforming DNA sequence comprises homology boxes without the presence of an incoming sequence. In this embodiment, it is desired to delete the endogenous DNA sequence between the two homology boxes. Furthermore, in some embodiments, the transforming sequences are wild-type, while in other embodiments, they are mutant or modified sequences. In addition, in some embodiments, the transforming sequences are homologous, while in other embodiments, they are heterologous.

As used herein, the term “target sequence” refers to a DNA sequence in the host cell that encodes the sequence where it is desired for the incoming sequence to be inserted into the host cell genome. In some embodiments, the target sequence encodes a functional wild-type gene or operon, while in other embodiments the target sequence encodes a functional mutant gene or operon, or a non-functional gene or operon.

As used herein, a “flanking sequence” refers to any sequence that is either upstream or downstream of the sequence being discussed (e.g., for genes A-B-C, gene B is flanked by the A and C gene sequences). In some embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), and in other embodiments, it is on each side of the sequence being flanked. The sequence of each homology box is homologous to a sequence in the Aspergillus chromosome. These sequences direct where in the Aspergillus chromosome the new construct gets integrated and what part of the Aspergillus chromosome will be replaced by the incoming sequence. In some embodiments these sequences direct where in the Aspergillus chromosome the new construct gets integrated without any part of the chromosome being replaced by the incoming sequence. In some embodiments, the 5′ and 3′ ends of a selective marker are flanked by a polynucleotide sequence comprising a section of the inactivating chromosomal segment. In some embodiments, a flanking sequence is present on only a single side (either 3′ or 5′), and in other embodiments, it is present on each side of the sequence being flanked.

As used herein, the term “chromosomally integrated” refers to a sequence, typically a mutant gene (e.g., disrupted form of a native gene), that has become incorporated into the chromosomal DNA of a host cell. Typically, chromosomal integration occurs via the process of “homologous recombination,” wherein the homologous regions of the introduced (transforming) DNA align with homologous regions of the host chromosome. Subsequently, the sequence between the homologous regions is replaced by the incoming sequence in a double crossover. Thus, “chromosomally integrated” is used interchangeably herein with “homologously recombined” or “homologously integrated.”

As used herein, the terms “selectable marker” and “selective marker” refer to a nucleic acid capable of expression in host cell, which allows for ease of selection of those hosts containing the marker. Thus, the term “selectable marker” refers to genes that provide an indication that a host cell has taken up (e.g., has been successfully transformed with) an incoming nucleic acid of interest (e.g., inactivated gene) or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation. Selective markers useful with the present invention include, but are not limited to, antimicrobial resistance markers (e.g., ampR; phleoR; specR; kanR; eryR; tetR; cmpR; hygroR and neoR; see e.g., Guerot-Fleury, Gene, 167:335-337 [1995]; Palmeros et al., Gene 247:255-264 [2000]; and Trieu-Cuot et al., Gene, 23:331-341 [1983]), auxotrophic markers, such as tryptophan, pyrG and amdS, and detection markers, such as β-galactosidase.

A “residing selectable marker” is a selectable marker that is located on the chromosome of the microorganism to be transformed. A residing selectable marker encodes a gene that is different from the selectable marker on the transforming DNA construct.

As used herein, the term “promoter” refers to a nucleic acid sequence that functions to direct transcription of a downstream gene. In some embodiments, the promoter is appropriate to the host cell in which a desired gene is being expressed. The promoter, together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “hybridization” refers to the process by which a strand of nucleic acid joins with a complementary strand through base pairing, as known in the art.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (50 below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while an intermediate or low stringency hybridization can be used to identify or detect polynucleotide sequence homologs.

Moderate and high stringency hybridization conditions are well known in the art. An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. An example of moderate stringent conditions include an overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denaturated sheared salmon sperm DNA, followed by washing the filters in 1×SSC at about 37-50° C. Those of skill in the art know how to adjust the temperature, ionic strength, etc. as necessary to accommodate factors such as probe length and the like.

As used herein, “recombinant” used in reference to a cell or vector refers to being modified by the introduction of a heterologous nucleic acid sequence, or a cell derived from a cell so modified. Thus, for example, recombinant cells express genes that are not found in identical form within the native (non-recombinant) form of the cell or express native genes that are otherwise abnormally expressed, underexpressed, overexpressed or not expressed at all as a result of deliberate human intervention. “Recombination, “recombining,” or generating a “recombined” nucleic acid is generally the assembly of two or more nucleic acid fragments wherein the assembly gives rise to a chimeric gene.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). Usually, the primer is single stranded for maximum efficiency in amplification. Most often, the primer is an oligodeoxyribonucleotide.

As used herein, the term “polymerase chain reaction” (“PCR”) refers to methods for amplifying DNA strands using a pair of primers, DNA polymerase, and repeated cycles of DNA polymerization, melting, and annealing (see, e.g., U.S. Pat. Nos. 4,683,195 4,683,202, and 4,965,188, which are hereby incorporated by reference herein).

As used herein, the terms “restriction endonucleases” and “restriction enzymes” refer to bacterial enzymes, each of which cut double-stranded DNA at or near a specific nucleotide sequence.

A “restriction site” refers to a nucleotide sequence recognized and cleaved by a given restriction endonuclease and is frequently the site for insertion of DNA fragments. In certain embodiments of the invention restriction sites are engineered into the selective marker and into 5′ and 3′ ends of the DNA construct.

### II. General Methods and Embodiments of the Inventions

The present invention provides inactivated mutants (e.g., deletion mutants and disruption mutants) that are capable of producing a protein of interest. In particular, the present invention relates to recombinant filamentous fungal microorganisms, such as Aspergillus species having altered expression of a protein of interest, wherein one or more chromosomal genes have been inactivated, and typically wherein one or more chromosomal genes have been deleted from the Aspergillus chromosome or wherein the protein-coding region of one or more chromosomal genes has been disrupted. Indeed, the present invention provides means for deletion of single or multiple genes. In some embodiments, such deletions provide advantages such as improved production of a protein of interest.

In some aspects, the present invention relies on routine techniques and methods used in the field of genetic engineering and molecular biology. The following resources include descriptions of general methodology useful in accordance with the invention: Sambrook et al., MOLECULAR CLONING: A LABORATORY MANUAL (2nd Ed., 1989); Kreigler, GENE TRANSFER AND EXPRESSION; A LABORATORY MANUAL (1990) and Ausubel et al., Eds. CURRENT PROTOCOLS IN MOLECULAR BIOLOGY (1994). These general references provide definitions and methods known to those in the art. However, it is not intended that the present invention be limited to any particular methods, protocols, and reagents described, as these may vary.

Inactivated Genes

As indicated above, the present invention includes filamentous fungal cells with single or multiple protease gene inactivations, wherein the inactivated genes are chosen from apsB, homologs of apsB, cpsA, homologs of cpsA, and combinations thereof. The genes may be inactivated using gene deletions or gene disruptions. In some embodiments, the inactivated genes are non-revertable.

In some embodiments, the inactivated gene is a homolog of apsB or a homolog of cpsA. Homologs useful with the present invention have the same or similar function as apsB or cpsA and share at least 99%, at least 98%, at least 97%, at least 96%, at least 95%, at least 94%, at least 93%, at least 92%, at least 91%, at least 90%, at least 88%, at least 85%, at least 80%, at least 70% or at least 60% sequence identity therewith.

The gene cpsA (SEQ ID NO: 1) encodes an A. niger carboxypeptidase enzyme having amino acid sequence SEQ ID NO:2, that are believed to be a secreted protein.

The gene apsB (SEQ ID NO: 9) encodes an A. niger aminopeptidase having amino acid sequence SEQ ID NO: 10, which is an intracellular protein (i.e., not secreted by the cell).

In some embodiments, the filamentous fungal cells may include additional inactivated genes. The additional inactivated genes may include but are not limited to those involved in protein degradation or protein modification, such as proteins in the ER degradation pathway, protease genes, such as secreted serine and aspartic protease genes, glycosylation genes and glycoprotein degradation genes. In some embodiments, the additional inactivated genes may be chosen from one or more of the following: derA, derB, htmA, mnn9, mnn10, ochA, dpp4, dpp5, pepF, pepAa, pepAb, pepAc and pepAd. The various coding sequences and functions of these genes, as well as the methods for making and using filamentous fungal cells having one or more of these genes inactivated are described in US patent application publication no. 2006/0246545, which is hereby incorporated by reference herein (see also, Wang et al., “Isolation of four pepsin-like protease genes from Aspergillus niger and analysis of the effect of disruptions on heterologous laccase expression,” Fungal Genet. Biol. 45(1): 17-27 (January 2008), which is hereby incorporated by reference herein).

In some embodiments, the filamentous fungal cells with inactivated genes of the present invention will include two or more (e.g. two, three or four) inactivated genes.

In some embodiments, the filamentous fungal cells may include additional inactivated genes. The additional inactivated genes may include but are not limited to those involved in protein degradation or protein modification, such as proteins in the ER degradation pathway, protease genes, such as secreted serine and aspartic protease genes, glycosylation genes and glycoprotein degradation genes. In some embodiments, the additional inactivated genes may be chosen from one or more of the following: derA, derB, htmA, mnn9, mnn10, ochA, dpp4, dpp5, pepF, pepAa, pepAb, pepAc and pepAd. The various coding sequences and functions of these genes, as well as the methods for making and using filamentous fungal cells having one or more of these genes inactivated are described in US patent application publication no. 2006/0246545, which is hereby incorporated by reference herein (see also, Wang et al., “Isolation of four pepsin-like protease genes from Aspergillus niger and analysis of the effect of disruptions on heterologous laccase expression,” Fungal Genet. Biol. 45(1): 17-27 (January 2008), which is hereby incorporated by reference herein).

In some embodiments, the filamentous fungal cells with inactivated genes of the present invention will include two or more (e.g. two, three or four) inactivated genes.

In some embodiment, the filamentous fungal cells with inactivated genes of the present invention include at least one gene chosen from apsB, homologs of apsB, cpsA, and homologs of cpsA, and a gene chosen from derA, derB, htmA, mnn9, mnn10, ochA, dpp4, dpp5, pepF, pepAa, pepAb, pepAc and pepAd, combinations thereof and functionally homologous sequences thereto having at least 99%, at least 98%, at least 97%, at least 96%, at least 95%, at least 94% at least 93%, at least 92%, at least 91%, at least 90%, at least 88%, at least 85%, at least 80%, at least 70% or at least 60% sequence identity therewith.

Further it is contemplated that the combinations of inactivated genes derA, derB, htmA, mnn9, mnn 10, ochA, dpp4, dpp5, pepF, pepAa, pepAb, pepAc and pepAd, specifically disclosed in US patent application publication no. 2006/0246545, can be combined with inactivated apsB and/or cpsA genes as disclosed herein, to provide filamentous fungal cells with two or more inactivated genes. In some embodiments, filamentous fungal cells may include inactivated dipeptidyl-protease genes, dpp4 and dpp5, in addition to the inactivated apsB and/or cpsA protease genes. In some embodiments, the filamentous fungal cells of the present invention may include one or more inactivated pepsin-like aspartic protease genes pepAa, pepAb, pepAc, and/or pepAd in addition to the inactivated apsB and/or cpsA protease genes. In some embodiment, the filamentous fungal cells with inactivated genes of the present invention include at least one gene chosen from apsB, homologs of apsB, cpsA, and homologs of cpsA, and a gene chosen from derA, derB, htmA, mnn9, mnn10, ochA, dpp4, dpp5, pepF, pepAa, pepAb, pepAc and pepAd, combinations thereof and functionally homologous sequences thereto having at least 99%, at least 98%, at least 97%, at least 96%, at least 95%, at least 94% at least 93%, at least 92%, at least 91%, at least 90%, at least 88%, at least 85%, at least 80%, at least 70% or at least 60% sequence identity therewith.

Further it is contemplated that the combinations of inactivated genes derA, derB, htmA, mnn9, mnn 10, ochA, dpp4, dpp5, pepF, pepAa, pepAb, pepAc and pepAd, specifically disclosed in US patent application publication no. 2006/0246545, can be combined with inactivated apsB and/or cpsA genes as disclosed herein, to provide filamentous fungal cells with two or more inactivated genes. In some embodiments, filamentous fungal cells may include inactivated dipeptidyl-protease genes, dpp4 and dpp5, in addition to the inactivated apsB and/or cpsA protease genes. In some embodiments, the filamentous fungal cells of the present invention may include one or more inactivated pepsin-like aspartic protease genes pepAa, pepAb, pepAc, and/or pepAd in addition to the inactivated apsB and/or cpsA protease genes.

In some embodiments, the homologous genes to apsB and cpsA found in filamentous fungal cells will find use in the present invention. In particular, the methods for making filamentous fungal cells with inactivated genes as disclosed herein may be used to inactivate mutant strains with these native homologs of apsB and/or cpsA inactivated. In some embodiments, these homologous genes for apsB and cpsA will have at least about 60%, 70%, 80%, 85%, 90%, 95%, or even greater percentage sequence identity to SEQ ID NO:1 (cpsA) or SEQ ID NO:8 (apsB), respectively.

Methods of Inactivation and DNA Constructs

Methods useful for identifying genes to inactivate in filamentous fungal cells (e.g., Aspergillus niger), for preparing DNA constructs for gene inactivation (e.g., disruption sequences), and for detecting gene inactivation are described in U.S. patent application publication no. 20060246545 A1, published Nov. 2, 2006, which is hereby incorporated by reference herein (see also, Wang et al., “Isolation of four pepsin-like protease genes from Aspergillus niger and analysis of the effect of disruptions on heterologous laccase expression,” Fungal Genet. Biol. 45(1): 17-27 (January 2008), which is hereby incorporated by reference herein).

Methods for determining homologous sequences from host cells are known in the art and include using a nucleic acid sequence disclosed herein to construct an oligonucleotide probe, said probe corresponding to about 6 to 20 amino acids of the encoded protein. The probe may then be used to clone the homologous gene. The filamentous fungal host genomic DNA is isolated and digested with appropriate restriction enzymes. The fragments are separated and probed with the oligonucleotide probe prepared from the protein degradation sequences by standard methods. A fragment corresponding to the DNA segment identified by hybridization to the oligonucleotide probe is isolated, ligated to an appropriate vector and then transformed into a host to produce DNA clones.

In some embodiments, a gene homolog of apsB or cpsA useful with the present invention can be a protein found in a filamentous fungal cell (e.g., Aspergillus sp.) having at least about 60%, 70%, 80%, 85%, 90%, 95%, or even greater percentage amino acid sequence identity to apsB (SEQ ID NO: 10) or cpsA (SEQ ID NO:2), respectively. In some embodiments, a functionally homologous nucleotide or amino acid sequence can be found in a related filamentous fungal species (e.g., Aspergillus niger and Aspergillus oryzae) and will have at least about 80%, 85%, 90%, or also at least 95% sequence identity apsB (SEQ ID NOS: 9 and 10) or cpsA (SEQ ID NOS: 1 and 2). In other embodiments, a gene homolog useful with the present invention can have a sequence resulting in an amino acid sequence differing from SEQ ID NOS: 10 or 2 by one or more conservative amino acid replacements. In such embodiments, the conservative amino acid replacements include but are not limited to the groups of glycine and alanine; valine, isoleucine and leucine; aspartic acid and glutamic acid; asparagine and glutamine; serine and threonine; tryptophan, tyrosine and phenylalanine; and lysine and arginine.

In some embodiments, the present invention includes a DNA construct comprising an incoming sequence (e.g., a disruption sequence). The DNA construct is assembled in vitro, followed by direct cloning of the construct into a competent host (e.g. an Aspergillus host), such that the DNA construct is integrated into the host chromosome. For example, PCR fusion and/or ligation can be employed to assemble a DNA construct in vitro.

In some embodiments, the DNA construct is a non-plasmid construct, while in other embodiments it is incorporated into a vector (e.g., a plasmid). In some embodiments, circular plasmids are used. In some embodiments, circular plasmids are designed to use an appropriate restriction enzyme (i.e., one that does not disrupt the DNA construct). Thus, linear plasmids find use in the present invention.

In some embodiments, the incoming sequence comprises an apsB gene, cpsA gene, homologous sequences to apsB or cpsA, gene fragments of apsB or cpsA; and/or immediate chromosomal coding region flanking sequences. A homologous sequence is a nucleic acid sequence encoding a protein having similar or identical function to apsB or cpsA, and having at least about 99%, 98%, 97%, 96%, 95%, 94% 93%, 92%, 91%, 90%, 88%, 85%, 80%, 70%, or 60% sequence identity to the apsB or cpsA gene or gene fragment thereof.

In some embodiments, wherein the genomic DNA is already known, the 5′ flanking fragment and the 3′ flanking fragment of the gene to be deleted is cloned by two PCR reactions, and in embodiments wherein the gene is disrupted, the DNA fragment is cloned by one PCR reaction.

In some embodiments, the coding region flanking sequences include a range of about 1 bp to 2500 bp; about 1 bp to 1500 bp, about 1 bp to 1000 bp, about 1 bp to 500 bp, and 1 bp to 250 bp. The number of nucleic acid sequences comprising the coding region flanking sequence may be different on each end of the gene coding sequence. For example, in some embodiments, the 5′ end of the coding sequence includes less than 25 bp and the 3′ end of the coding sequence includes more than 100 bp.

In some embodiments, the incoming sequence comprises is a disruption sequence that comprises a selective marker flanked on the 5′ and 3′ ends with a fragment of the gene sequence. In other embodiments, when the DNA construct comprising the selective marker and gene, gene fragment or homologous sequence thereto is transformed into a host cell, the location of the selective marker renders the gene non-functional for its intended purpose. In some embodiments, the incoming sequence comprises the selective marker located in the promoter region of the gene. In other embodiments, the incoming sequence comprises the selective marker located after the promoter region of gene.

In yet other embodiments, the incoming sequence is a disruption sequence comprising the selective marker located in the coding region of the gene. In further embodiments, the incoming sequence comprises a selective marker flanked by a homology box on both ends. In still further embodiments, the incoming sequence includes a sequence that interrupts the transcription and/or translation of the coding sequence. In yet additional embodiments, the DNA construct includes restriction sites engineered at the upstream and downstream ends of the construct.

In one embodiment, the A. nidulans amdS gene provides a selectable marker system for the transformation of filamentous fungi useful with the present invention. The amdS gene codes for an acetamidase enzyme deficient in strains of Aspergillus and provides positive selective pressure for transformants grown on acetamide media. The amdS gene can be used as a selectable marker even in fungi known to contain an endogenous amdS gene or homolog, e.g., in A. nidulans (Tilburn et al. 1983, Gene 26: 205-221) and A. oryzae (Gomi et al. 1991, Gene 108: 91-98). Background amdS activity of non-transformants can be suppressed by the inclusion of CsCl in the selection medium.

Methods for using amdS marker system in the transformation of industrially important filamentous fungi are established in the art (e.g., in Aspergillus niger (see e.g., Kelly and Hynes 1985, EMBO J. 4: 475-479; Wang et al., Fungal Genet Biol. 45(1): 17-27 (January 2008)); in Penicillium chrysogenum (see e.g., Beri and Turner 1987, Curr. Genet. 11: 639-641); in Trichoderma reesei (see e.g., Pentilla et al. 1987, Gene 61: 155-164); in Aspergillus oryzae (see e.g., Christensen et al. 1988, Bio/technology 6: 1419-1422); in Trichoderma harzianum (see e.g., Pe'er et al. 1991, Soil Biol. Biochem. 23: 1043-1046); and U.S. Pat. No. 6,548,285, each of which is hereby incorporated by reference herein).

The DNA constructs comprising an incoming sequence may be incorporated into a vector (e.g., in a plasmid), or used directly to transform the filamentous fungal cell, thereby resulting in an inactivated mutant. Typically, the DNA construct is stably transformed resulting in chromosomal integration of the inactivated gene which is non-revertable. Methods for in vitro construction and insertion of DNA constructs into a suitable vector are well known in the art. Deletion and/or insertion of sequences is generally accomplished by ligation at convenient restriction sites. If such sites do not exist, synthetic oligonucleotide linkers can be prepared and used in accordance with conventional practice. (See, Sambrook (1989) supra, and Bennett and Lasure, MORE GENE MANIPULATIONS IN FUNGI, Academic Press, San Diego (1991) pp 70-76.). Additionally, vectors can be constructed using known recombination techniques (e.g., Invitrogen Life Technologies, Gateway Technology). Examples of suitable expression and/or integration vectors that may be used in the practice of the invention are provided in Sambrook et al., (1989) supra, Ausubel (1987) supra, van den Hondel et al. (1991) in Bennett and Lasure (Eds.) MORE GENE MANIPULATIONS IN FUNGI, Academic Press pp. 396-428 and U.S. Pat. No. 5,874,276. Exemplary vectors useful with the present invention include pBS-T, pFB6, pBR322, pUC18, pUC100 and pENTR/D.

In some embodiments, at least one copy of a DNA construct is integrated into the host chromosome. In some embodiments, one or more DNA constructs of the invention are used to transform host cells. For example, one DNA construct may be used to inactivate an apsB gene and another construct may be used to inactivate a cpsA gene. Of course, additional combinations are contemplated and provided by the present invention.

Inactivation occurs via any suitable means, including deletions, substitutions (e.g., mutations), disruptions, insertions in the nucleic acid gene sequence, and/or gene silencing mechanisms, such as RNA interference (RNAi). In one embodiment, the expression product of an inactivated gene is a truncated protein with a corresponding change in the biological activity of the protein. In some embodiments, the inactivation results in a loss of biological activity of the gene. In some embodiments, the biological activity of the inactivated gene in a recombinant fungal cell will be effectively zero (i.e., unmeasurable). In some embodiments, some residual activity may remain, and often will be less than 25%, 20%, 15%, 10%, 5%, and 2%, or less compared to the biological activity of the same or homologous gene in a corresponding parent strain.

In some embodiments, inactivation is achieved by deletion and in other embodiments inactivation is achieved by disruption of the protein-coding region of the gene. In some embodiments, the gene is inactivated by homologous recombination.

In some embodiments, the deletion may be partial as long as the sequences left in the chromosome render the gene functionally inactive. In some embodiments, a deletion mutant comprises deletion of one or more genes that results in a stable and non-reverting deletion. Flanking regions of the coding sequence may include from about 1 bp to about 500 bp at the 5′ and 3′ ends. The flanking region may be larger than 500 bp but typically does not include other genes in the region which may be inactivated or deleted according to the invention. The end result is that the deleted gene is effectively non-functional. While not meant to limit the methods used for inactivation in some embodiments, apsB and/or cpsA and homologous genes may be inactivated by deletion.

In some embodiments, the disruption sequence comprises an insertion of a selectable marker gene into the protein-coding region. Typically, this insertion is performed in vitro by reversely inserting a gene sequence into the coding region sequence of the gene inactivated by cleaving then ligating at a restriction site. Flanking regions of the coding sequence may include about 1 bp to about 500 bp at the 5′ and 3′ ends. The flanking region may be larger than 500 bp, but will typically not include other genes in the region. The DNA construct aligns with the homologous sequence of the host chromosome and in a double crossover event the translation or transcription of the gene is disrupted. For example, the apsB chromosomal gene is aligned with a plasmid comprising the gene or part of the gene coding sequence and a selective marker. In some embodiments, the selective marker gene is located within the gene coding sequence or on a part of the plasmid separate from the gene. The vector is chromosomally integrated into the host, and the host's gene is thereafter inactivated by the presence of the marker inserted in the coding sequence.

While not meant to limit the methods used for inactivation, in some embodiments apsB and/or cpsA and homologous sequences may be inactivated by this method.

In some embodiments, inactivation of the gene is by insertion in a single crossover event with a plasmid as the vector. For example, the vector is integrated into the host cell chromosome and the gene is inactivated by the insertion of the vector in the protein-coding sequence of the gene or in the regulatory region of the gene.

In alternative embodiments, inactivation results due to mutation of the gene. Methods of mutating genes are well known in the art and include but are not limited to site-directed mutation, generation of random mutations, and gapped-duplex approaches (See e.g., U.S. Pat. No. 4,760,025; Moring et al., Biotech. 2:646 [1984]; and Kramer et al., Nucleic Acids Res., 12:9441 [1984].

Host Filamentous Fungal Cells

In the present invention, the host cell can be a filamentous fungal cell (See, Alexopoulos, C. J. (1962), INTRODUCTORY MYCOLOGY, Wiley, New York). The type of filamentous fungal cell is not critical. Filamentous fungal cells useful with the present invention include, but are not limited to: Aspergillus sp., (e.g., A. oryzae, A. niger, A. awamori, A. nidulans A. sojae, A. japonicus, A. kawachi and A. aculeatus); Rhizopus sp., Trichoderma sp. (e.g., Trichoderma reesei (previously classified as T. longibrachiatum and currently also known as Hypocrea jecorina), Trichoderma viride, Trichoderma koningii, and Trichoderma harzianums)) and Mucor sp. (e.g., M. miehei and M. pusillus). In some embodiments, the host cells are Aspergillus niger cells.

In some embodiments, the present invention may be used with particular strains of Aspergillus niger include ATCC 22342 (NRRL 3112), ATCC 44733, and ATCC 14331 and strains derived there from. In some embodiments, the host cell is capable of expressing a heterologous gene. For example, the host cell may be a recombinant cell, which produces a heterologous protein. In other embodiments, the host is one that overexpresses a protein that has been introduced into the cell.

In some embodiments, the host strain is a mutant strain deficient in one or more genes such as genes corresponding to protease genes other than the apsB and cpsA genes. For example, it is contemplated that a Aspergillus niger host cell may be used in which a gene encoding the major secreted aspartyl protease, such as aspergillopepsin has been deleted (see e.g., U.S. Pat. Nos. 5,840,570 and 6,509,171, which are hereby incorporated by reference herein). Thus, the present invention provides for apsB and/or cpsA inactivated mutant strains of filamentous fungal cells, wherein the corresponding parent strain already is an inactivated mutant with one or more inactivated genes.

Proteins of Interest

In some embodiments an inactivated mutant encompassed by the invention will exhibit altered expression and translation (i.e., protein production) of one or more endogenous and/or heterologous proteins of interest in comparison to the expression and translation of the same protein(s) by the corresponding parent strain of filamentous fungus.

In some embodiments, the inactivated mutants of filamentous fungal cells encompassed by the invention will produce the endogenous and/or heterologous proteins of interest in an amount at least about 0% to about 200% (or more) greater than the production of the same protein(s) in the corresponding parent strain. Accordingly, in some embodiments, the production of the protein(s) of interest by the inactivated mutant is at least about 0% to 100% greater, and in some embodiments is at least about 10% to 60% greater, including embodiments wherein production at least about 10%, 15%, 20%, 25%, 30%, 35%, 40%, 45%, 50%, and 55% greater, than the production of the endogenous and/or heterologous protein(s) in the corresponding parent strain.

In alternative embodiments, it is desired to have decreased production of a protein of interest. Accordingly, the present invention also provide an inactivated mutant of filamentous fungal cell wherein production of an endogenous and/or heterologous protein(s) of interest is at least about 0% to 100%, or even less than the production of the endogenous and/or heterologous protein in a corresponding parent strain of the filamentous fungus. In some embodiments, the production of the protein is at least about 10% to 60% less, including embodiments wherein production at least about 10%, 15%, 20%, 25%, 30%, 35%, 40%, 45%, 50%, and 55% less than the production of the endogenous and/or heterologous protein(s) in the corresponding parent strain.

In some embodiments of the present invention, the protein of interest produced by the inactivated mutant of a filamentous fungal cell is an intracellularly produced protein (i.e., an intracellular, non-secreted polypeptide). In other embodiments, the protein of interest is a secreted polypeptide. In addition, the protein of interest may be a fusion or hybrid protein. In some embodiments, the inactivated mutant exhibits altered production of a plurality of proteins, some of which are intracellular and some of which are secreted.

Proteins of interest useful with the present invention include enzymes known in the art, including, but not limited to those chosen from amylolytic enzymes, proteolytic enzymes, cellulytic enzymes, oxidoreductase enzymes and plant cell-wall degrading enzymes. More particularly, these enzyme include, but are not limited to amylases, glucoamylases, proteases, xylanases, lipases, laccases, phenol oxidases, oxidases, cutinases, cellulases, hemicellulases, esterases, perioxidases, catalases, glucose oxidases, phytases, pectinases, glucosidases, isomerases, transferases, galactosidases and chitinases. In some embodiments, enzymes include but are not limited to amylases, glucoamylases, proteases, phenol oxidases, cellulases, hemicellulases, glucose oxidases and phytases. In some embodiments, the polypeptide of interest is a protease, cellulase, glucoamylase or amylase.

For example, in one embodiment of the present invention inactivation of apsB in A. niger results in increased production of a heterologous laccase enzyme as well as endogenous glucogenic enzymes.

In some embodiments, the protein of interest is a secreted polypeptide, which is fused to a signal peptide (i.e., an amino-terminal extension on a protein to be secreted). Nearly all secreted proteins use an amino-terminal protein extension, which plays a role in the targeting to and translocation of precursor proteins across the membrane. This extension is proteolytically removed by a signal peptidase during or immediately following membrane transfer.

In some embodiments of the present invention, the polypeptide of interest is a protein such as a protease inhibitor, which inhibits the action of proteases. Protease inhibitors are known in the art, for example the protease inhibitors belonging to the family of serine proteases inhibitors which are known to inhibit trysin, cathepsinG, thrombin and tissue kallikrein. Among the protease inhibitors useful in the present invention are Bowman-Birk inhibitors and soybean trypsin inhibitors (See, Birk, Int. J. Pept. Protein Res. 25:113-131 [1985]; Kennedy, Am. J. Clin. Neutr. 68:1406 S-1412S [1998] and Billings et al., Proc. Natl. Acad. Sci. 89:3120-3124 [1992]).

In some embodiments of the present invention, the polypeptide of interest is chosen from hormones, antibodies, growth factors, receptors, cytokines, etc. Hormones encompassed by the present invention include but are not limited to, follicle-stimulating hormone, luteinizing hormone, corticotropin-releasing factor, somatostatin, gonadotropin hormone, vasopressin, oxytocin, erythropoietin, insulin and the like. Growth factors include, but are not limited to platelet-derived growth factor, insulin-like growth factors, epidermal growth factor, nerve growth factor, fibroblast growth factor, transforming growth factors, cytokines, such as interleukins (e.g., IL-1 through IL-13), interferons, colony stimulating factors, and the like. Antibodies include but are not limited to immunoglobulins obtained directly from any species from which it is desirable to produce antibodies. In addition, the present invention encompasses modified antibodies. Polyclonal and monoclonal antibodies are also encompassed by the present invention. In some embodiments, the antibodies or fragments thereof are chimeric or humanized antibodies, including but not limited to: anti-p185^{Her2}, HulD10-, trastuzumab, bevacizumab, palivizumab, infliximab, daclizumab, and rituximab.

In a further embodiment, the nucleic acid encoding the protein of interest will be operably linked to a suitable promoter, which shows transcriptional activity in a fungal host cell. The promoter may be derived from genes encoding proteins either endogenous or heterologous to the host cell. The promoter may be a truncated or hybrid promoter. Further the promoter may be an inducible promoter. Typically, the promoter is useful in a Trichoderma host or an Aspergillus host. Suitable nonlimiting examples of promoters include cbh1, cbh2, eg/1, eg/2, and xyn1. In one embodiment, the promoter is one that is native to the host cell. Other examples of useful promoters include promoters from the genes of A. awamori and A. niger glucoamylase genes (glaA) (Nunberg et al., (1984) Mol. Cell. Biol. 4:2306-2315 and Boel et al., (1984) EMBO J. 3:1581-1585); Aspergillus oryzae TAKA amylase; Rhizomucor miehei aspartic proteinase; Aspergillus niger neutral alpha-amylase; Aspergillus niger acid stable alpha-amylase; Trichoderma reesei stp1 and the cellobiohydrolase 1 gene promoter (see e.g., EP 0 137 280 A1, which is hereby incorporated by reference herein) and mutant, truncated and hybrid promoters thereof.

In some embodiments, the polypeptide coding sequence is operably linked to a signal sequence which directs the encoded polypeptide into the cell's secretory pathway. The 5′ end of the coding sequence may naturally contain a signal sequence naturally linked in translation reading frame with the segment of the coding region which encodes the secreted polypeptide. The DNA encoding the signal sequence typically is the sequence which is naturally associated with the polypeptide to be expressed. Typically, the signal sequence is encoded by an Aspergillus niger alpha-amylase, Aspergillus niger neutral amylase or Aspergillus niger glucoamylase. In some embodiments, the signal sequence is the Trichoderma cdh1 signal sequence which is operably linked to a cdh1 promoter.

Transformation of Filamentous Fungal Cells

Introduction of a DNA construct or vector into a host cell includes techniques such as transformation; electroporation; nuclear microinjection; transduction; transfection, (e.g., lipofection mediated and DEAE-Dextrin mediated transfection); incubation with calcium phosphate DNA precipitate; high velocity bombardment with DNA-coated microprojectiles; agrobacterium mediated transformation and protoplast fusion. General transformation techniques are known in the art (see, e.g., Ausubel et al., (1987), supra, chapter 9; and Sambrook (1989) supra, Campbell et al., (1989) Curr. Genet. 16:53-56 and THE BIOTECHNOLOGY OF FILAMENTOUS FUNGI, Chap. 6. Eds. Finkelstein and Ball (1992) Butterworth and Heinenmann, each of which is hereby incorporated by reference herein).

Production of heterologous proteins in filamentous fungal cell expression systems are also known in the art. For example, the expression of heterologous proteins in Trichoderma is described in Harkki et al. (1991); Enzyme Microb. Technol. 13:227-233; Harkki et al., (1989) Bio Technol. 7:596-603; EP 244,234; EP 215,594; and Nevalainen et al., “The Molecular Biology of Trichoderma and its Application to the Expression of Both Homologous and Heterologous Genes”, in MOLECULAR INDUSTRIAL MYCOLOGY, Eds. Leong and Berka, Marcel Dekker Inc., NY (1992) pp. 129-148; and U.S. Pat. Nos. 6,022,725 and 6,268,328, each of which is hereby incorporated by reference herein.

The expression of heterologous proteins in Aspergillus sp. is described in Cao et al., (2000) Sci. 9:991-1001; and U.S. Pat. No. 6,509,171, each of which is hereby incorporated by reference herein.

Transformants of the present invention can be purified using known techniques.

Methods for Detecting Gene Inactivation

One skilled in the art may use various methods to determine if a gene has been inactivated. While not meant to limit the invention one method which can be used is the phenol/chloroform method described in Zhu (Zhu et al., Acat Mycologica Sinica 13:34-40 [1994], which is hereby incorporated by reference herein. Briefly, in this method the genomic DNA is used as a template for PCR reactions. Primers are designed so that one primer anneals to a selectable marker gene (e.g., the amdS gene) and a second primer anneals to a sequence further 3′ from the DNA homologous fragment at the 3′ end of the gene. An inactivated mutant will produce a specific PCR product when its genomic DNA is used as a PCR reaction template as opposed to the corresponding parent strain (having a non-inactivated gene) which will not generate PCR fragments when its genomic DNA is used as a template. In addition the PCR fragment from the inactivated mutant may be subjected to DNA sequencing to confirm the identity if the inactivated gene. Other useful methods include Southern analysis and reference is made to Sambrook (1989) supra.

Cell Culture

The filamentous fungal cells may be grown in conventional culture medium. The culture media for transformed cells may be modified as appropriate for activating promoters and selecting transformants. The specific culture conditions, such as temperature, pH and the like will be apparent to those skilled in the art. Typical culture conditions for filamentous fungi useful with the present invention are well known and may be found in the scientific literature such as Sambrook, (1982) supra, and from the American Type Culture Collection. Additionally, fermentation procedures for production of heterologous proteins are known per se in the art. For example, proteins can be produced either by solid or submerged culture, including batch, fed-batch and continuous-flow processes. Fermentation temperature can vary somewhat, but for filamentous fungi such as Aspergillus niger the temperature generally will be within the range of about 20° C. to 40° C., typically in the range of about 28° C. to 37° C., depending on the strain of microorganism chosen. The pH range in the aqueous microbial ferment (fermentation admixture) should be in the exemplary range of about 2.0 to 8.0. With filamentous fungi, the pH normally is within the range of about 2.5 to 8.0; with Aspergillus niger the pH normally is within the range of about 4.0 to 6.0, and typically in the range of about 4.5 to 5.5. While the average retention time of the fermentation admixture in the fermentor can vary considerably, depending in part on the fermentation temperature and culture employed, generally it will be within the range of about 24 to 500 hours, typically about 24 to 400 hours. Any type of fermentor useful for culturing filamentous fungi may be employed in the present invention. One useful embodiment with the present invention is operation under 15L Biolafitte (Saint-Germain-en-Laye, France).

Methods for Determining Expressed Protein Activity

Various assays are known to those of ordinary skill in the art for detecting and measuring activity of intracellularly and extracellularly expressed polypeptides. Means for determining the levels of secretion of a protein of interest in a host cell and detecting expressed proteins include the use of immunoassays with either polyclonal or monoclonal antibodies specific for the protein. Examples include enzyme-linked immunosorbent assay (ELISA), radioimmunoassay (RIA), fluorescence immunoassay (FIA), and fluorescent activated cell sorting (FACS). However, other methods are known to those in the art and find use in assessing the protein of interest (See e.g., Hampton et al., SEROLOGICAL METHODS, A LABORATORY MANUAL, APS Press, St. Paul, Minn. [1990]; and Maddox et al., J. Exp. Med., 158:1211 [1983], each of which is hereby incorporated by reference herein). In some embodiments, the expression and/or secretion of a protein of interest are enhanced in an inactivated mutant. In some embodiments the production of the protein of interest is at least 100%, at least 95%, at least 90%, at least 80%, at least 70%, at least 60%, at least 50%, at least 40%, at least 30%, at least 20%, at least 15%, at least 10%, at least 5% and at least 2% greater in the inactivated mutant as compared to the corresponding parent strain.

Protein Recovery

Once the desired protein is expressed and, optionally, secreted the protein of interest may be recovered and further purified. The recovery and purification of the protein of interest from a fermentation broth can be done by procedures known in the art. The fermentation broth will generally contain cellular debris, including cells, various suspended solids and other biomass contaminants, as well as the desired protein product.

Suitable processes for such removal include conventional solid-liquid separation techniques such as, e.g., centrifugation, filtration, dialysis, microfiltration, rotary vacuum filtration, or other known processes, to produce a cell-free filtrate. Often, it may be useful to further concentrate the fermentation broth or the cell-free filtrate prior to crystallization using techniques such as ultrafiltration, evaporation or precipitation.

Precipitating the proteinaceous components of the supernatant or filtrate may be accomplished by means of a salt, followed by purification by a variety of chromatographic procedures, e.g., ion exchange chromatography, affinity chromatography or similar art recognized procedures. When the expressed desired polypeptide is secreted the polypeptide may be purified from the growth media. Typically, the expression host cells are removed from the media before purification of the polypeptide (e.g., by centrifugation).

When the expressed recombinant desired polypeptide is not secreted from the host cell, usually the host cell is disrupted and the polypeptide released into an aqueous “extract” which is the first stage of purification. Typically, the expression host cells are collected from the media before the cell disruption (e.g., by centrifugation).

The manner and method of carrying out the present invention may be more fully understood by those of skill in the art by reference to the following examples, which examples are not intended in any manner to limit the scope of the present invention or of the claims directed thereto.

### EXAMPLES

The following Examples are provided in order to demonstrate and further illustrate specific embodiments and aspects of the present invention and are not to be construed as limiting the scope thereof.

In the experimental disclosure which follows, the following abbreviations apply: ° C. (degrees Centigrade); H₂O (water); dH₂O (deionized water); HCl (hydrochloric acid); aa (amino acid); bp (base pair); kb (kilobase pair); kD (kilodaltons); g (grams); μg (micrograms); mg (milligrams); μl (microliters); ml (milliliters); mm (millimeters); μm (micrometer); M (molar); mM (millimolar); μM (micromolar); MW (molecular weight); s (seconds); min(s) (minute/minutes); hr(s) (hour/hours); NaCl (sodium chloride); PBS (phosphate buffered saline [150 mM NaCl, 10 mM sodium phosphate buffer, pH 7.2]); PCR (polymerase chain reaction); SDS (sodium dodecyl sulfate); w/v (weight to volume); v/v (volume to volume); ATCC (American Type Culture Collection, Rockville, Md.); BD BioSciences (Previously CLONTECH Laboratories, Palo Alto, Calif.); Invitrogen (Invitrogen Corp., San Diego, Calif.); and Sigma (Sigma Chemical Co., St. Louis, Mo.).

### Example 1

### Aspergillus niger Cells Having Inactivated cpsA Gene

a. Preparation of “disruption plasmid” having inactivated cpsA DNA construct

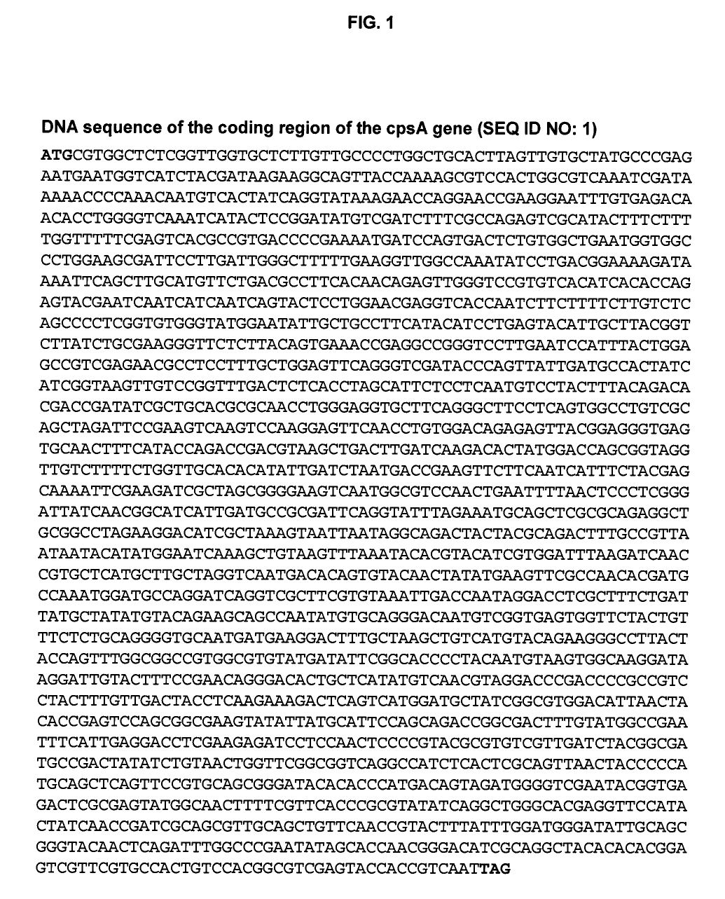

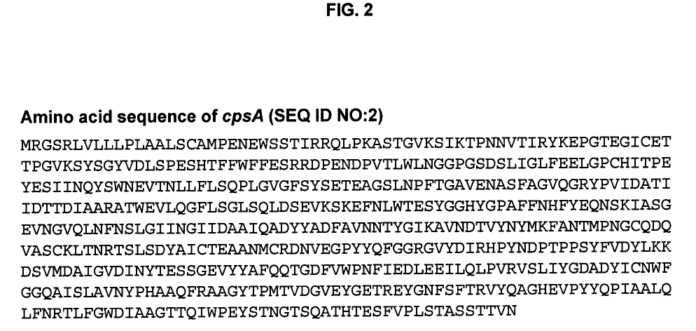

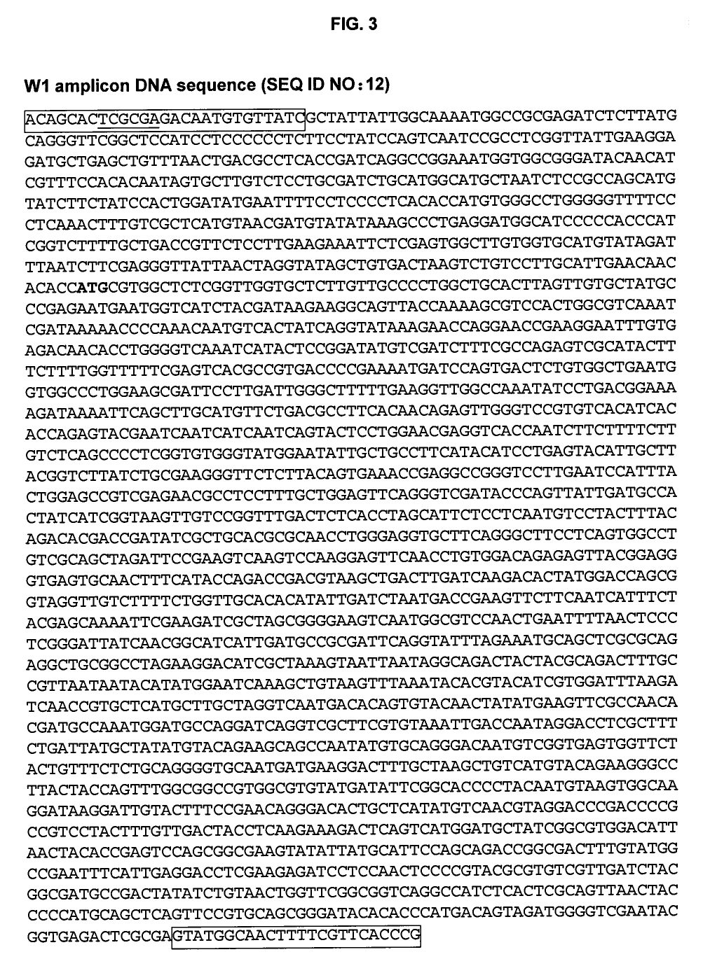

FIG. 1 depicts the 2188 bp genomic DNA sequence of the Aspergillus cpsA gene (SEQ ID NO: 1). FIG. 2 depicts the 552 amino acid sequence (SEQ ID NO: 2) encoded by the cpsA genomic DNA sequence of SEQ ID NO:1. Carboxypeptidases are proteases that cleave amino acids from the C-terminal end of a polypeptide.

The cpsA “disruption plasmid” is the DNA construct comprising the inactivated cpsA gene that is used to transform the A. niger cells and thereby generate the cpsA inactivated mutant microorganism. The general strategy and methods used to make the cpsA (and apsB) disruption plasmid used herein were the same as described in U.S. patent application publication no. 20060246545 A1, published Nov. 2, 2006, which is hereby incorporated by reference herein (see also, Wang et al., “Isolation of four pepsin-like protease genes from Aspergillus niger and analysis of the effect of disruptions on heterologous laccase expression,” Fungal Genet. Biol. 45(1): 17-27 (January 2008), which is hereby incorporated by reference herein).

Table 1 below depicts the primer sequences used to make the inactivated cpsA disruption plasmid construct and to detect the disrupted gene construct in the transformed cells.

TABLE 1

| | |
|---|---|
| Primer | |
| Name | Primer Sequence (5′ to 3′) |

| | | |
|---|---|---|
| Pa | ACAGCACTCGCGAGACAATGTGTTATC | (SEQ ID NO: 3) |
| Ta | CGGGTGAACGAAAAGTTGCCATAC | (SEQ ID NO: 4) |
| P_{amdS} | TTTCCAGTCTAGACACGTATAACGGC | (SEQ ID NO: 5) |
| P_{P-out}a | ACGGAGTCGGACCAAGACACTAAG | (SEQ ID NO: 6) |

The primer denoted “Pa” (SEQ ID NO: 3) amplifies the promoter region of the cpsA gene. The primer denoted “Ta” (SEQ ID NO: 4) amplifies the terminator region of cpsA. Using these primers, δ 2495 bp fragment was amplified under the following PCR conditions: (1) the PCR tube was heated at 94° C. for 4 min to denature template DNA, (2) the PCR reaction was then run at 94° C. for 1 min, 60° C. for 2 min, and 72° C. for 2 min 30 sec, and (3) this cycle was repeated 30 times. The PCR reaction was extended at 72° for 10 min before the tube was incubated at 4° C. This amplicon, named W1, includes the 1986 bp coding region and 509 bp promoter region of cpsA, and is depicted in FIG. 3 (SEQ ID NO: 7).

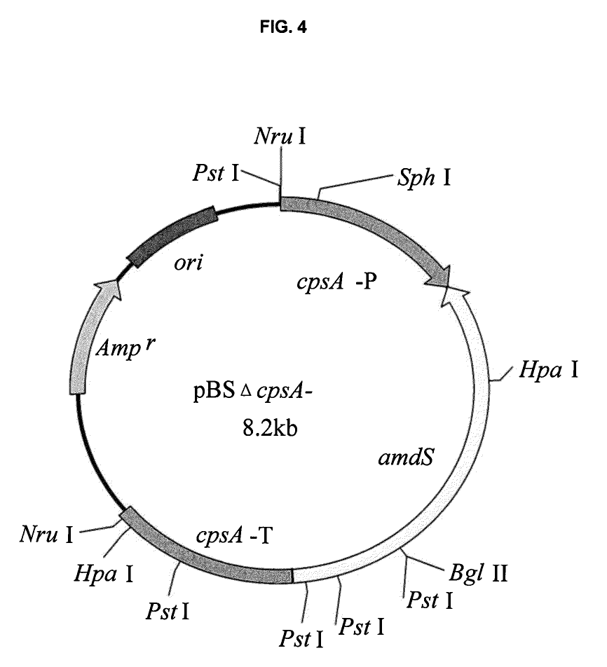

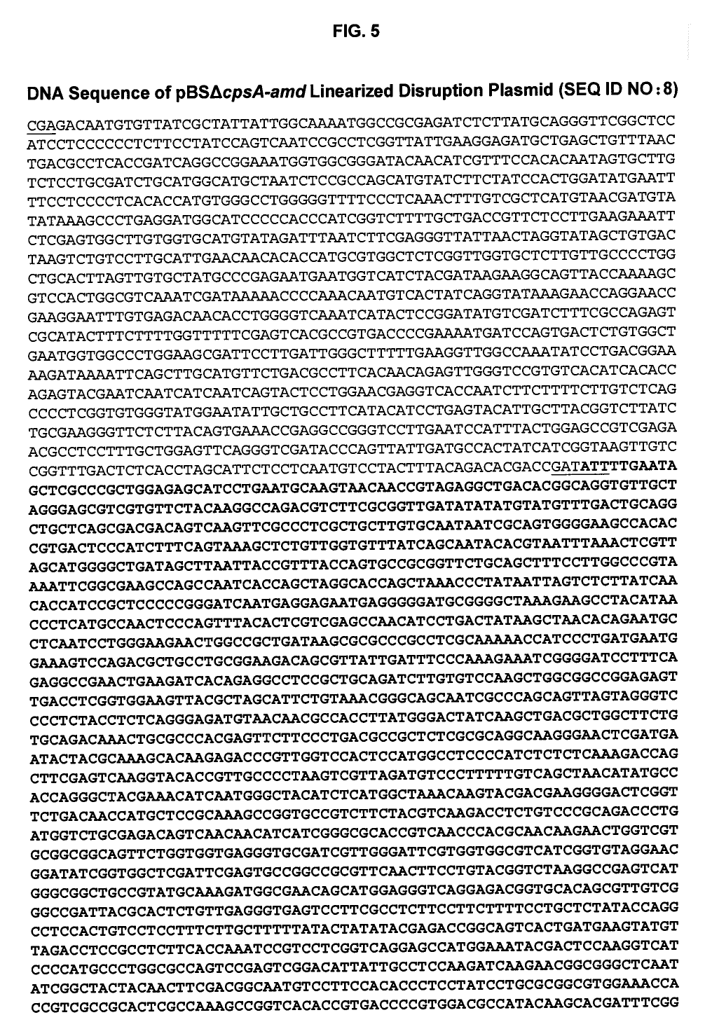

The W1 amplicon was cloned into pBS-T, a TA vector derived from pBlue-script (Tian Wei Biotech. Co. Ltd) to construct plasmid, pBS-W1 (cpsA). A 2.7 kb DNA fragment containing the A. nidulans amdS expression cassette was inserted reversely at a unique EcoRV site in the coding region of the cpsA gene to generate the disruption plasmid, pBSΔcpsA-amd depicted by the plasmid map in FIG. 4.

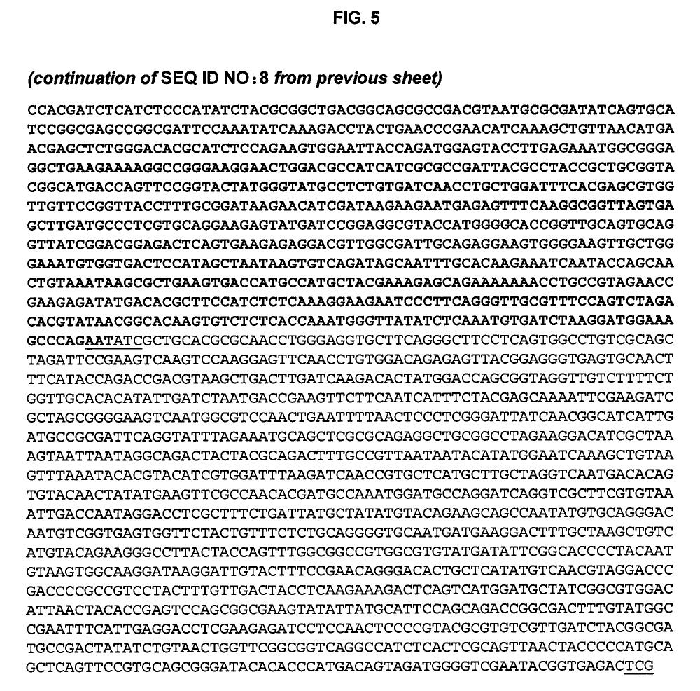

The pBSΔcpsA-amd plasmid was linearized by Nrul restriction enzyme digestion resulting in the linearized disruption plasmid fragment (i.e., “disruption sequence”) having the DNA sequence shown in FIG. 5 (SEQ ID NO: 8).

b. Transformation of A. niger with Linearized Disruption Plasmid

The linearized pBSΔcpsA-amd disruption sequence (SEQ ID NO:8) was used to transform recipient strain GICC2773, which is a derivative of an AP-4 Aspergillus niger strain (see Ward et al., Appli. Microbiol. Biotechnol 39:738-743 (1993)). GICC2773 includes a disruption mutant of the pepA gene and an integrated plasmid expressing a heterologous enzyme, laccase (Icc1 of the Tramete versicolor laccase gene) under glucoamylase promoter and terminator control. The GICC2773 strain is described in greater detail by Valkonen et al., “Improvement of foreign-protein production in Aspergillus niger var. awamori by constitutive induction of the unfolded-protein response,” Appl. Environ. Microbiol. 69(12); 6979-6986 (2003), which is hereby incorporated by reference herein.

The transformation protocol utilized was a modification of the Campbell method (see, Campbell et at., Curr. Genet. 16:53-56 (1989), which is hereby incorporated by reference herein) with beta-D-glucinase G (InterSpex Products, Inc. San Mateo, Calif.) used to produce protoplasts and the pH adjusted to 5.5. Briefly, protoplast preparation and A. niger transformation were carried out as follows:
(a) a 1-2 ml spore suspension made from fresh slant culture was inoculated into 50 ml liquid medium (Soluble starch 3%, yeast extract 2%, KH₂PO₄ 0.5%, corn meal 0.5%, Natural pH), in a shake flask and was cultivated on a rotor shaker at 200 rpm, 30° C. for 13-14 hr;
(b) mycelium was collected by filtrating culture through gauze and washed three times with water, once with 0.8M MgSO₄ (pH 5.8).
(c) washed mycelium were placed into 100 ml flask, suspended in 15 ml 0.8M MgSO₄ containing 150 mg of lysing enzyme (Sigma-Aldrich, St. Louis, Mo.) and 15 mg of cellulase R-10 (Yakult Biochemical Co., Ltd., Nishinomiya, Japan);
(d) the mycelium cell wall was digested at 30° C. for 1-2 hrs which flask shaken at 80 rpm, and protoplast formation was monitored under microscope;
(e) protoplasts were harvested from cell lysate by filtering through two layers of 200 mesh nylon membrane to remove cell debris;
(f) protoplasts were collected and washed with sorbitol solution (1.2 M sorbitol, 50 mM CaCl₂, 10 mM Tris pH 7.4) two times by centrifuge at 700 g for 6-8 min;
(g) protoplasts were resuspended in 200 μl of sorbitol solution and were counted with a blood counter to determine concentration;
(h) 10 μg transformation DNA (i.e., linearized pBSΔcpsA-amd disruption plasmid) was mixed with 2−4×10⁷ protoplast;
(i) to the above mixture, 50 μl of PEG6000 (or PEG4000) solution (PEG 50%, 50 mM CaCl₂, 10 mM Tris pH 7.4) were added and mixed gently but thoroughly, and put on ice for 30 min;
(j) 1 ml PEG solution was added, mixed well, and placed at room temperature for 20 min;
(k) 1 ml sorbitol solution was added and mixed well with 56-58° C. molten soft agar and then the whole mixture immediately was poured onto transformation medium plate;
(l) the plate was incubated at 30° C. for 4-8 days.

All solutions and media were either autoclaved or filter sterilized through a 0.2 micron filter.

c. Detection of A. niger Inactivated cpsA Mutant

DNA was extracted from randomly picked transformants using the phenol/chloroform method (see, Zhu et al., Nucleic Acid Res. 21:5279-80 (1993), which is hereby incorporated by reference herein).

Successfully transformed colonies were detected by PCR amplification using primers P_{amdS} (SEQ ID NO: 5) and P_{P-out}a (SEQ ID NO: 6). These primers were designed to generate a 1378 bp amplicon from the genomic DNA when the linearized disruption plasmid was homologously recombined into the A. niger genome.

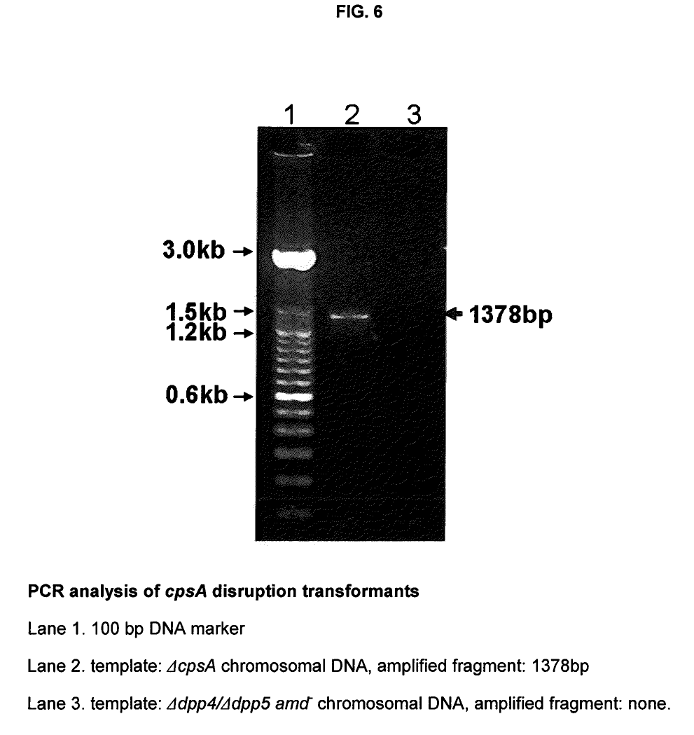

As shown in FIG. 6, PCR was used to identify a successfully transformed A. niger strain, ΔcpsA, which exhibited the predicted 1378 bp amplicon. As predicted, the non-transformed A. niger parent strain failed to show a PCR product.

Sequencing of the 1378 bp amplicon also confirmed that it was the inactivated cpsA gene.

d. Production of Heterologous Laccase in cpsA Inactivated A. niger

As noted above, the corresponding parent strain GICC2773 includes an integrated plasmid expressing a heterologous enzyme, laccase from Tramete versicolor, under glucoamylase promoter and terminator control. The level of heterologous laccase production by the inactivated strain, ΔcpsA was assayed relative to the corresponding parent strain.

Laccase activity was assayed following a standard procedure based on the oxidation of ABTS (2,2′-azino-bis(3-ethylbenzothiazoline-6-sulfonate) by oxygen in pH 4.6 sodium acetate buffer (Sigma-Aldrich, St. Louis, Mo.). The strains were first cultured in 250 ml baffled flasks containing 50 mL of Promosoy growth media (Central Soya, Fort Wayne, Ind.) suitable for laccase production. The inactivated and non-inactivated (i.e., corresponding parent strain) A. niger strains were grown in shake flasks for 120 hrs. The culture supernatants were obtained by centrifugation to remove mycelium. The supernatant, which contains laccase proteins, was incubated with ABTS in the sodium acetate buffer at 37° C. for 30 min and color formation was measured as increased optical density at 420 nM. In addition, total extracellular protein was measured using the Folin phenol method (see Lowry, et al., [1951] J. Biol. Chem. 193:265-275).

The cpsA inactivated mutant strain, ΔcpsA, showed a 14.2% decrease in laccase production in comparison to the non-inactivated GICC2773 parent strain, and a 35.7% increase in mycelium dry weight percentage of the mutant. Thus, heterologous laccase production was altered negatively by the inactivation of carboxypeptidase gene, cpsA. However, it is possible that other heterologous (or endogenous) proteins that were not assayed here, are exhibiting (or would exhibit) increased production due to the cpsA inactivation.

### Example 2

### Aspergillus niger Cells Having Inactivated apsB Gene

Unless otherwise noted, the same methods used in Example 1 were used to prepare an A. niger strain having inactivated apsB gene.

a. Preparation of “Disruption Plasmid” Having Inactivated apsB DNA Construct

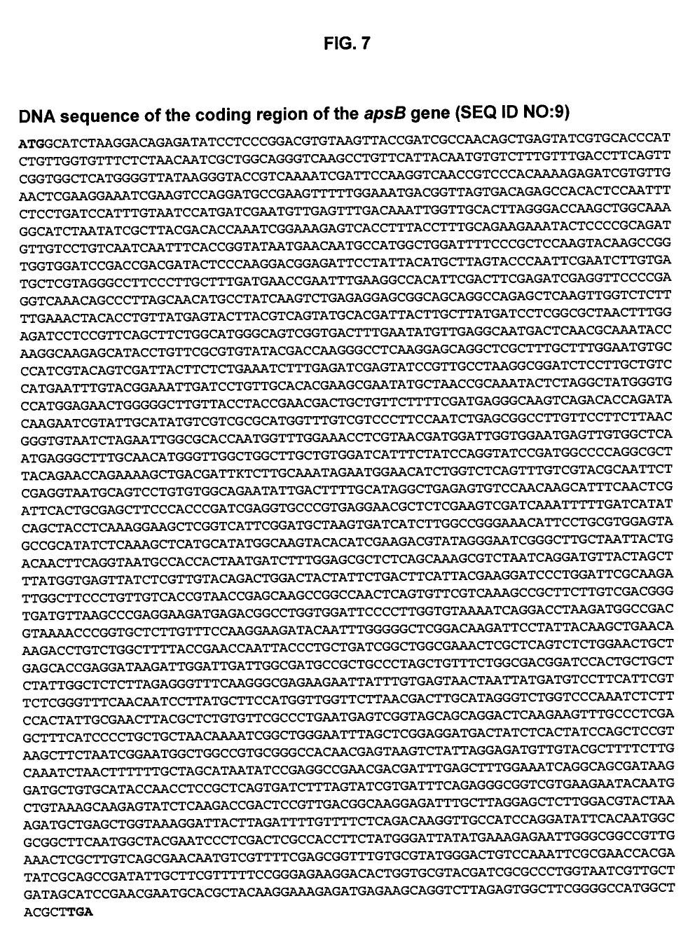

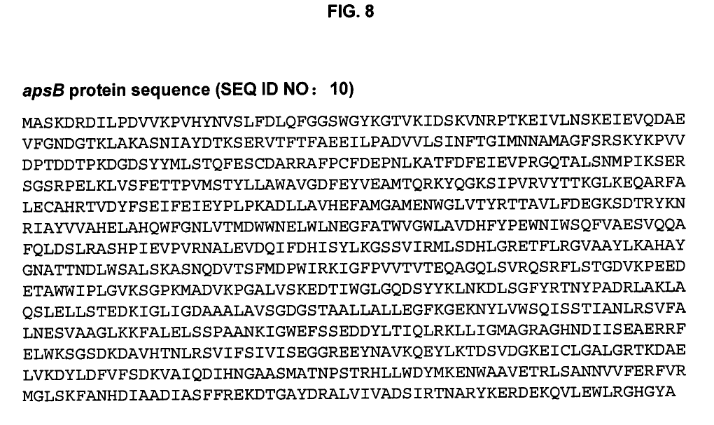

FIG. 7 depicts the 3352 bp genomic DNA sequence of the Aspergillus niger aminopeptidase gene, apsB (SEQ ID NO: 9). FIG. 8 depicts the 881 amino acid sequence of the translated apsB protein (SEQ ID NO: 10). Aminopeptidases are proteases that remove amino acids from the N-terminal end of a polypeptide. The product of apsB is an intracellular enzyme (i.e., non-secreted protein) and consequently its activity is expected to be limited to affecting proteins within the cell.

Table 2 below depicts the primer sequences used to make the inactivated apsB disruption plasmid constructs.

TABLE 2

| | |
|---|---|
| Primer | |
| Name | Sequence (5′ to 3′) |

| | | |
|---|---|---|
| Pb | ACCCGACGTGGTGGTATGAATGCTC | (SEQ ID NO: 11) |
| Tb | AGGTGGCGAGTCGAGGGATTCGTAG | (SEQ ID NO: 12) |
| P_{P-out}b | ACCGTAGGTAGGCAGACTTGGCTCC | (SEQ ID NO: 13) |

The PCR primer used to amplify the promoter region is designated in Table 2 as “Pb” (SEQ ID NO: 11), and the primer used to amplify the terminator region is designated “Tb” (SEQ ID NO: 12). Using these primers, the 3078 bp coding region of the 3370 bp apsB gene and 222 bp promoter region were amplified.

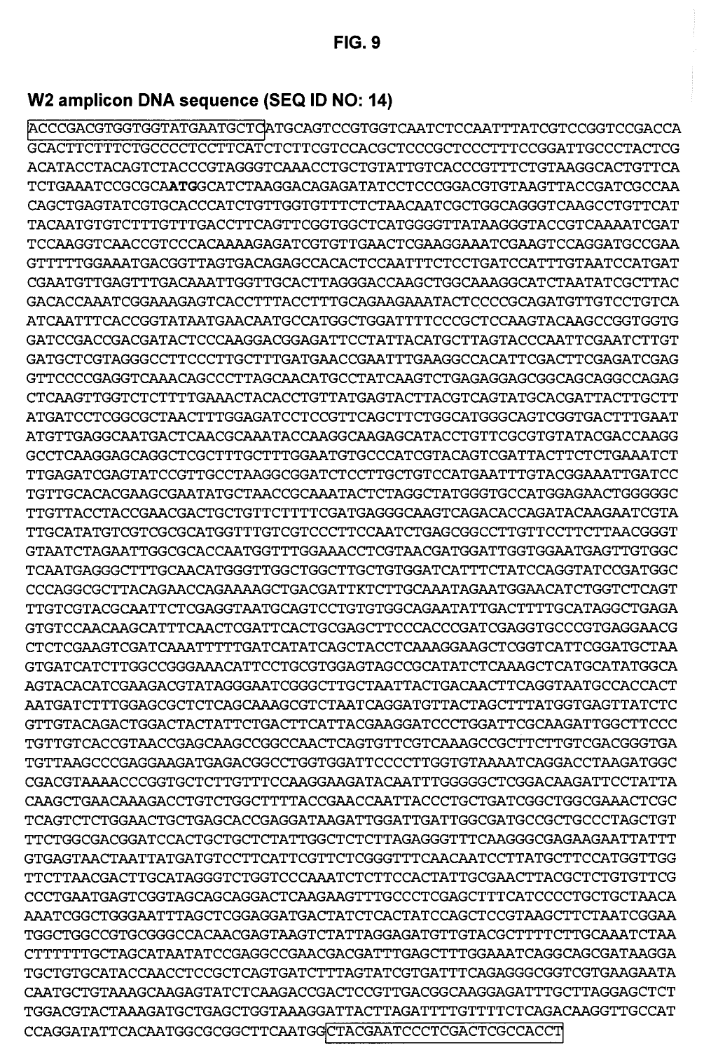

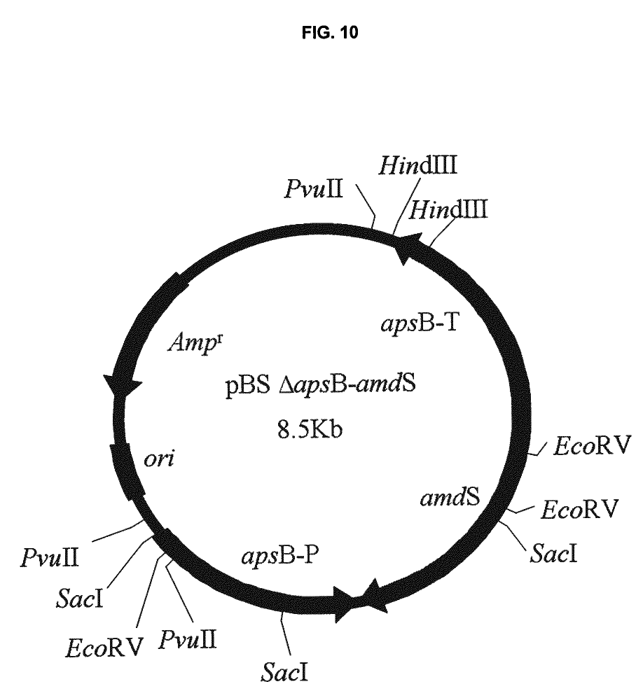

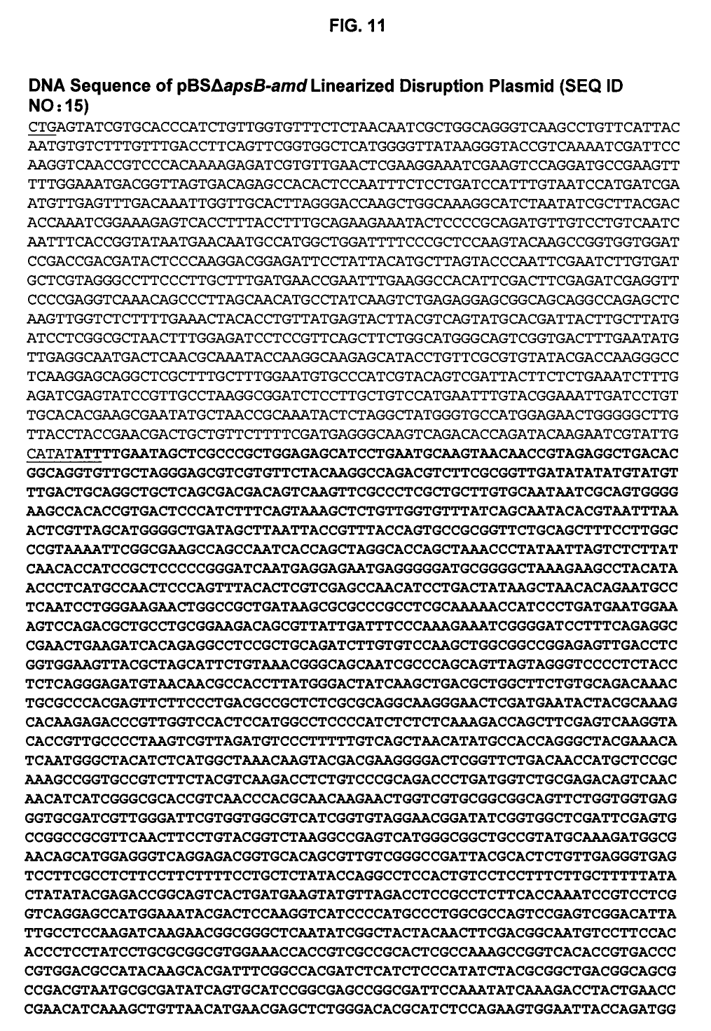

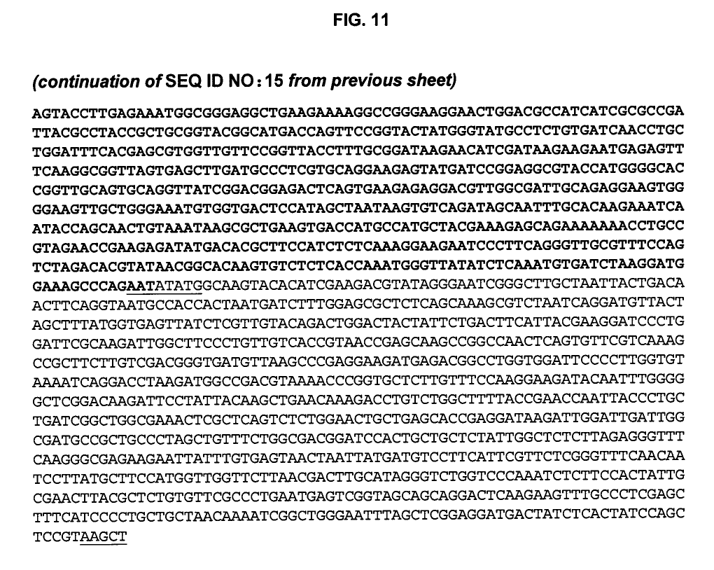

The 3300 bp PCR amplicon, W2, whose sequence is depicted in FIG. 9 (SEQ ID NO: 14), was cloned into a pBS-T vector (Tian Wei Biotech Co. Ltd.) to construct apsB plasmid pBS-W2(apsB). The DNA fragment containing the A. nidulans gene, amdS was inserted into the coding region of the apsB gene at the NdeI-NdeI (1628-2168 bp) site to generate the apsB disruption plasmid, pBSΔapsB-amdS depicted in FIG. 10. The plasmid was linearized by restriction enzyme digestion (HindIII and PvuII resulting in the linearized disruption plasmid DNA fragment (i.e., “disruption sequence”) having the sequence shown in FIG. 11 (SEQ ID NO: 15).

b. Transformation of A. niger with Linearized Disruption Plasmid

The linearized pBSΔapsB-amdS disruption sequence (SEQ ID NO: 15) was used to transform the A. niger GICC2773 strain and genomic DNA was extracted from the transformants, as described above for Example 1.

c. Detection of A. niger Inactivated apsB Mutant

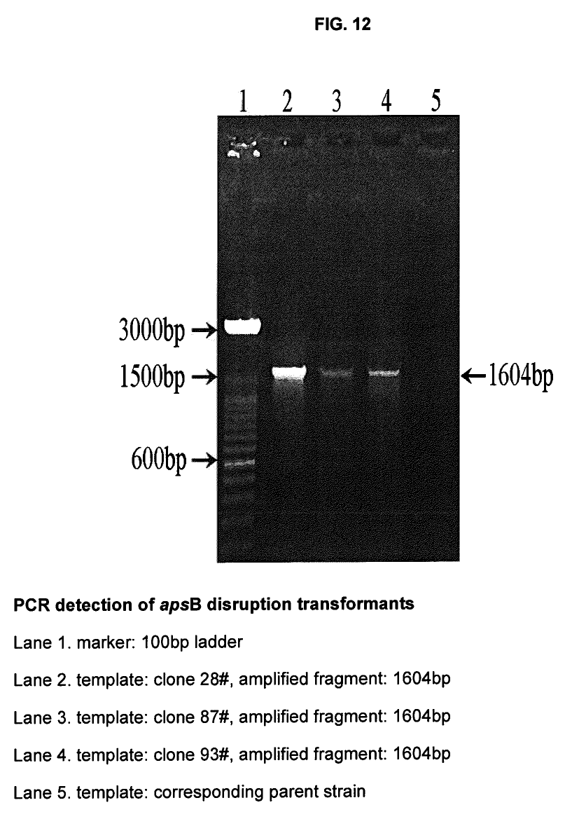

A total of 90 transformants were randomly picked. Clones successfully transformed with pBSΔapsB-amdS were detected by PCR using two primers P_{amdS} (SEQ ID NO: 5) and P_{T-out}b (SEQ ID NO: 13). These primers were designed to result in a specific 1604 bp amplicon when genomic DNA from the inactivated strain was used as template for PCR amplification. As shown in FIG. 12, gel electrophoresis of chromosomal DNA identified three clones (#28, #87, and #93) that exhibited the 1604 bp amplicon indicating successful transformation with the inactivated apsB gene. No 1604 bp amplicon was observed when the DNA was from the A. niger corresponding parent strain not transformed with the disruption plasmid. Sequencing of the 1604 bp amplicon further confirmed the result.

d. Production of Heterologous Laccase in apsB Inactivated A. niger

The three apsB inactivated mutant clones #28, #87, and #93 were assayed for heterologous laccase activity as described in Example 1 and the results are shown in Table 3 below.

TABLE 3

| Inactivated | | mycelium dry weight |
|---|---|---|
| Mutant Strain | Production of Laccase | (% compared to |
| ΔapsB clone | (% increase in OD420) | parent strain) |
| clone#28 | 25.8 | 84.3 |
| clone#87 | 22.5 | 85.2 |
| clone#93 | 21.5 | 84.5 |

Each of the three clones showed a significant percentage increase (21.5% to 25.8%) in laccase production and concomitant 15% decrease in mycelium dry weight percentage relative to the non-inactivated parent strain. Thus, the inactivation of the intracellular protease apsB results in a significant enhancement of the filamentous fungal cells of A. niger to produce the heterologous gene product laccase.

### Example 3

### Aspergillus niger Cells Having Triple Gene Inactivations

The production of a double mutant strain of A. niger, Δdpp4/Δdpp5 amd, that has two inactivated dipeptidyl peptidase genes, dpp4 and dpp5 disrupted by the amdS selectable marker, was described in U.S. patent application publication no. 20060246545 A1, published Nov. 2, 2006, which is hereby incorporated by reference herein. This double-inactivation strain was used as a starting material to prepare triple-inactivation mutants of A. niger as described below.

a. Inactivated cpsA, dpp4 and dpp5

The cpsA disruption plasmid constructed and linearized as shown in Example 1 was used to transform the double-inactivation A. niger strain (Δdpp4/Δdpp5 amd) which expresses a Tramete laccase under the glucoamylase promoter and terminator control.

The triple inactivation strain resulting from successful transformation was detected by PCR using three pairs of primers—one pair for each of the three inactivated genes. As in Example 1, above, the primer pair of SEQ ID NOS: 5 and 6 was used to detect the single cpsA disruption. The primer pairs used to detect the double-inactivated strain Δdpp4/Δdpp5 amd were SEQ ID NOS: 37 and 67 and SEQ ID NOS: 64 and 96 disclosed in U.S. patent application publication no. 20060246545 A1, which is hereby incorporated by reference herein.

One clone (#44) was isolated from the resulting triple disruption strain ΔcpsA/Δdpp41Δdpp5 and assayed for heterologous laccase production (based on laccase activity assay as in Example 1) and mycelium dry weight relative to the parent Δdpp4/Δdpp5 amd strain. As shown in Table 4, the triple inactivated strain exhibited decreased laccase activity (8.4%) and a slight increase in total dry cell weight (104.8%).

TABLE 4

| | | mycelium dry weight % |
|---|---|---|
| Inactivated | Production of Laccase | (compared to parent |
| Mutant strain (Δ) | (% Increase in OD420) | strain) |
| ΔcpsA/Δdpp4/ | −8.4 | 104.8 |
| Δdpp5 clone #44 | | |
| ΔapsB/Δdpp4/ | −23.4 | 98.6 |
| Δdpp5 | | |

b. Inactivated apsB, dpp4 and dpp5 Genes.

As described above in Example 3a, the triple inactivated mutant strain with inactivated apsB was prepared by using the apsB disruption plasmid, constructed and linearized in Example 2, to transform the double-inactivated A. niger strain (Δdpp4/Δdpp5 amd), prepared as described in U.S. patent application publication no. 20060246545 A1, published Nov. 2, 2006, which is hereby incorporated by reference herein.

The successfully transformed triple inactivated A. niger strain ΔapsB/Δdpp4/Δdpp5 was detected by PCR using three pairs of primers. The primer pair used to confirm the presence of the apsB disruption was SEQ ID NO: 5 and 13 (as in Example 2). The primer pairs used to detect the double-inactivated strain Δdpp4/Δdpp5 amd were SEQ ID NOS: 37 and 67 and SEQ ID NOS: 64 and 96 disclosed in U.S. patent application publication no. 20060246545 A1, which is hereby incorporated by reference herein.

The triple inactivation strain ΔapsB/Δdpp41Δdpp5 was isolated and assayed for heterologous laccase production using the laccase activity assay as in Example 1, and total mycelium dry weight relative to the corresponding parent Δdpp4/Δdpp5 amd strain. As shown in Table 4, the ΔapsB/Δdpp4/Δdpp5 strain exhibited decreased laccase activity (23.4%) and a slightly decreased total dry cell weight (98.6%).

### Example 4

### Increased Native Glucogenic Enzyme Production in apsB Inactivated A. niger

In order to determine the effect of apsB inactivation on production of an endogenous enzyme, the three mutant clones described in Example 2 (clones #28, #87, and #93) were assayed as for total glucogenic enzyme activity in comparison to the corresponding parent strain, GICC2773. The measured total glucogenic enzyme activity represents a measure of the expression of native glucoamylase by the inactivated strains, although the assay actually measures a composite of the activity of several glucogenic enzymes.

The assay was carried out as described by Wang et al., (Fungal Genet. Biol. 45(1): 17-27 (January 2008), which is hereby incorporated by reference herein) with the exception that different inactivated mutant A. niger strains were assayed.

ΔapsB clones #28, #87, and #93, and corresponding parent strain GICC2773 were grown in shake-flasks in S3Y2 medium at 30° C. and 200 rpm. After 120 hours, 1 ml of the culture was centrifuged and a 60 μl sample of culture filtrate supernatant collected. Each 60 μl sample was mixed with 140 μl H₂O and 2.8 ml 2% soluble starch substrate (in 0.1 M HOAc buffer at pH 4.65). After incubating at 37° C. for 30 minutes, the reaction was stopped by boiling. A 20 μl aliquot of the reaction solution was mixed with 2 ml glucose oxidase/peroxidase reagent from a commercial glucose assay kit (Zhongsheng Biotechnology Co. Ltd., Beijing, China). Liberated glucose was determined by measuring the optical density at 525 nm according to the glucose assay kit. The total glucogenic enzyme activity was calculated as international units of enzyme activity secreted per gram dry weight of mycelium (IU/g).

The results of the above assay were as follows: clones #28, #87, and #93, exhibited increased total glucogenic enzyme activity (relative to GICC2773 parent strain) of 81.0%, 40.5%, and 71.7%, respectively. Thus, the inactivation of the intracellular protease apsB in A. niger resulted in substantial increases in native glucogenic enzyme activity.

Those of skill in the art readily appreciate that the present invention is well adapted to carry out the objects and obtain the ends and advantages mentioned, as well as those inherent therein. The compositions and methods described herein are representative, exemplary embodiments, and are not intended as limitations on the scope of the invention.

While particular embodiments of the present invention have been illustrated and described, it will be apparent to those skilled in the art that various other changes and modifications can be made without departing from the spirit and scope of the invention. It is therefore intended to cover in the appended claims all such changes and modifications that are within the scope of this invention.

The invention illustratively described herein suitably may be practiced in the absence of any element(s) or limitation(s) which is not specifically disclosed herein. The terms and expressions which have been employed are used as terms of description and not of limitation, and there is no intention that in the use of such terms and expressions of excluding any equivalents of the features shown and described or portions thereof.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All patents and publications are herein incorporated by reference to the same extent as if each individual publication was specifically and individually indicated to be incorporated by reference.

What is claimed is:

1. A filamentous fungal cell comprising at least one inactivated gene, wherein the inactivated gene is chosen from apsB, a homolog of apsB, cpsA, a homolog of cpsA, and combinations thereof.

2. The filamentous fungal cell of claim 1, wherein the inactivated gene is a homolog of cpsA, wherein the homolog has at least 85% sequence identity to SEQ ID NO: 1.

3. The filamentous fungal cell of claim 1, wherein the inactivated gene is a homolog of apsB, wherein the homolog has at least 85% sequence identity to SEQ ID NO: 9.

4. The filamentous fungal cell of claim 1, wherein said filamentous fungus is chosen from: Aspergillus sp., Rhizopus sp., Trichoderma sp., and Mucor sp.

5. The filamentous fungal cell of claim 4, wherein said filamentous fungus is of Aspergillus sp. chosen from: of A. oryzae, A. niger, A. awamori, A. nidulans, A. sojae, A. japonicus, A. kawachi and A. aculeatus.

6. The filamentous fungal cell of claim 5, wherein the filamentous fungus is A. niger.

7. The filamentous fungal cell of claim 1, wherein the inactivated gene is apsB (SEQ ID NO: 9).

8. The filamentous fungal cell of claim 7, wherein the filamentous fungal cell comprises at least a second inactivated gene chosen from: cpsA, dpp4, dpp5, and homologs thereof.

9. The filamentous fungal cell of claim 1, wherein the inactivated gene is cpsA (SEQ ID NO: 1).

10. The filamentous fungal cell of claim 9, wherein the filamentous fungal cell comprises at least a second inactivated gene chosen from: apsB, dpp4, dpp5, and homologs thereof.

11. The filamentous fungal cell of claim 1, wherein the inactivated gene is inactivated by disruption with a selectable marker gene.

12. The filamentous fungal cell of claim 1, wherein the production of an endogenous protein by the cell is at least about 10% to about 60% greater than the production of the endogenous protein in a corresponding parent strain of the filamentous fungal cell.

13. The filamentous fungal cell of claim 12, wherein the endogenous protein is a glucogenic enzyme.

14. The filamentous fungal cell of claim 12, wherein the endogenous protein is an enzyme chosen from: α-amylase, cellulase, glucoamylase, laccase, neutral proteases, and alkaline protease.

15. The filamentous fungal cell of claim 1, wherein the cell further comprises a nucleic acid encoding a heterologous protein.

16. The filamentous fungal cell of claim 15, wherein the production of the heterologous protein is altered relative to the production of the same protein in a corresponding parent strain of the filamentous fungal cell.

17. The filamentous fungal cell of claim 15, wherein the production of the heterologous protein is at least about 10% to about 60% greater than the production of the same protein in a corresponding parent strain of the filamentous fungal cell.

18. The filamentous fungal cell of claim 15, wherein the total dry cell weight differs by less than about 25%, 20%, 15%, 10%, or 5% less than the total dry cell weight of a corresponding parent strain of the filamentous fungal cell.

19. The filamentous fungal cell of claim 15, wherein the heterologous protein is an enzyme.

20. The filamentous fungal cell of claim 19, wherein the enzyme is chosen from: α-amylase, cellulase, glucoamylase, laccase, neutral proteases, and alkaline protease.

21. The filamentous fungal cell of claim 19, wherein the enzyme is a laccase.

22. The filamentous fungal cell of claim 15, wherein the heterologous protein is a protease inhibitor

23. The filamentous fungal cell of claim 15, wherein the heterologous protein is an antibody or fragment thereof.

24. The filamentous fungal cell of claim 1, wherein said inactivated gene encodes an intracellular protein.

25. The filamentous fungal cell of claim 24, wherein said intracellular protein is a protease.

26. The filamentous fungal cell of claim 1, wherein said filamentous fungal cell comprises at least two inactivated genes.

27. The filamentous fungal cell of claim 1, further comprising an inactivated gene chosen from:
derA, derB, htmA, mnn9, mnn 10, ochA, dpp4, dpp5, pepAa, pepAb, pepAc, pepAd, pepB, pepC, pepD, pepF, and homologs thereto.

28. A filamentous fungal cell comprising at least one inactivated gene, wherein the inactivated gene encodes an intracellular protein, and wherein production of at least one other protein by the cell is at least about 10% to about 60% greater than the production of the other protein in a corresponding parent strain of the filamentous fungal cell.

29. The filamentous fungal cell of claim 28, wherein the intracellular protein is a protease.

30. The filamentous fungal cell of claim 28, wherein the intracellular protein is an aminopeptidase.

31. The filamentous fungal cell of claim 28, wherein the intracellular protein is apsB.

32. A method for producing a protein comprising:
a) introducing a nucleic acid encoding a protein into a filamentous fungal cell, wherein said cell comprises at least one inactivated gene chosen from: apsB, a homolog of apsB, cpsA, a homolog cpsA, and combinations thereof; and
b) growing the cell under conditions suitable for producing the protein.

33. The method according to claim 32, wherein the method further comprises recovering the protein.

34. The method according to claim 32, wherein the inactivated gene is a homolog of cpsA, wherein the homolog has at least 85% sequence identity to SEQ ID NO: 1.

35. The method according to claim 32, wherein the inactivated gene is a homolog of apsB, wherein the homolog has at least 85% sequence identity to SEQ ID NO: 9.

36. The method according to claim 32, wherein the inactivated gene is cpsA (SEQ ID NO: 1).

37. The method according to claim 32, wherein the inactivated gene is apsB (SEQ ID NO: 9).

38. The method according to claim 32, wherein said filamentous fungus is of Aspergillus sp. chosen from: A. oryzae, A. niger, A. awamori, A. nidulans, A. sojae, A. japonicus, A. kawachi and A. aculeatus.

39. The method according to claim 38, wherein the Aspergillus sp. is A. niger.

40. The method according to claim 32, wherein the protein is a protease inhibitor.

41. The method according to claim 32, wherein the protein is an antibody or fragment thereof.

42. The method according to claim 32, wherein the protein is an enzyme.

43. A method for making a filamentous fungal strain for protein production comprising:
a) transforming a filamentous fungal cell with a disruption sequence, wherein the disruption sequence comprises at least one inactivated gene chosen from: apsB, a homolog of apsB, cpsA, a homolog cpsA, and combinations thereof; and
b) selecting the transformed cells wherein said disruption sequence is chromosomally integrated.

44. The method according to claim 43, wherein the inactivated gene is a homolog of cpsA, wherein the homolog has at least 85% sequence identity to SEQ ID NO: 1.

45. The method according to claim 43, wherein the inactivated gene is a homolog of apsB, wherein the homolog has at least 85% sequence identity to SEQ ID NO: 9.

46. The method according to claim 43 wherein the filamentous fungus is chosen from: Aspergillus sp., Rhizopus sp., Trichoderma sp., and Mucor sp.

47. The method according to claim 43, wherein the filamentous fungus is Aspergillus sp. chosen from: A. oryzae, A. niger, A. awamori, A. nidulans, A. sojae, A. japonicus, A. kawachi and A. aculeatus.

48. The method according to claim 47, wherein the filamentous fungus is A. niger.

49. The method according to claim 43, wherein the inactivated gene is apsB (SEQ ID NO: 9).

50. The method according to claim 49, wherein the filamentous fungal cell comprises at least a second inactivated genes chosen from: cpsA, dpp4, dpp5, and homologs thereof.

51. The method according to claim 43, wherein the inactivated gene is cpsA (SEQ ID NO: 1).

52. The method according to claim 51, wherein the filamentous fungal cell comprises at least a second inactivated genes chosen from: apsB, dpp4, dpp5, and homologs thereof.

53. The method according to claim 43, wherein the cell further comprises a nucleic acid encoding a heterologous protein.

54. The method according to claim 43, wherein the disruption sequence comprises a selectable marker gene sequence reversely inserted at a restriction site in the coding region sequence of the inactivated gene.

55. The method according to claim 54, wherein the selectable marker gene is amdS.

56. An isolated nucleic acid comprising a disruption sequence of a gene, wherein the gene is cpsA or a homolog of cpsA and wherein the disruption sequence comprises a selectable marker gene sequence reversely inserted at a restriction site in the coding region sequence of the gene.

57. The isolated nucleic acid of claim 56, wherein gene is a homolog of cpsA having at least 85% sequence identity to SEQ ID NO: 1.

58. The isolated nucleic acid of claim 56, wherein gene is cpsA (SEQ ID NO: 1).

59. The isolated nucleic acid of claim 56, wherein the selectable marker gene is amdS.

60. A vector comprising the nucleic acid of claim 56.

61. An isolated nucleic acid comprising a disruption sequence of a gene, wherein the gene is apsB or a homolog of cpsA and wherein the disruption sequence comprises a selectable marker gene sequence reversely inserted at a restriction site in the coding region sequence of the gene.

62. The isolated nucleic acid of claim 61, wherein gene is a homolog of apsB having at least 85% sequence identity to SEQ ID NO: 9.

63. The isolated nucleic acid of claim 61, wherein gene is apsB (SEQ ID NO: 9).

64. The isolated nucleic acid of claim 61, wherein the selectable marker gene is amdS.

65. A vector comprising the nucleic acid of claim 61.
